SEQUENCE LISTING (2 sequences)

SEQ ID NO: 1 - DNA, 3393 bp, Artificial Sequence
  Artificially synthesized expression cassette plasmid
ggccgccaccgctgagcaataactagcataaccccttggggcctctaaacgggtcttgaggggttttttgctgaaaggaggaactatatccgggtaacgaattcaagcttgatatcattcaggacgagcctcagactccagcgtaactggactgcaatcaactcactggctcaccttcacgggtgggcctttcttcggtagaagtcatctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgcgcagaaaggcccacccgaaggtgagccaggtgattacatttgggccctcattagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatagctcctgaaaatctcgataactcaaaaaatacgcccggtagtgatcttatttcattatggtgaaagttggaacctcttacgtgccgatcaaaaagacggtcaaaagcctccggtcggaggcttttgactttctgctatggaggtcaggtatgatttaaatggtcagtattgagcgatatctagagaattcgtccaccacaattcagcaaattgtgaacatcatcacgttcatctttccctggttgccaatggcccattttcctgtcagtaacgagaaggtcgcgaattcaggcgctttttagactggtcgtaatgaaattctttttaagaggagatatacataggtctcacatgattaaattaaaatttggtgttttttttacagttttactatcttcagcatatgcacatggaacacctcaaaatattactcacggtgtgttaaccctgtcttctcgtggtctggactatccatacgatgtaccggattacgcgcgttatggaagacgagtgttaacaatggagatcttcgaacaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagcttaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttcggttagagc

SEQ ID NO: 2 - protein, 9 aa, Haemophilus influenzae
YPYDVPDYA

(19) United States
(12) Patent Application Publication
(10) Pub. No.: US 2022/0362361 A1
(43) Pub. Date: Nov. 17, 2022

(54) COMPOSITIONS AND METHODS FOR PRODUCING ENHANCED IMMUNE RESPONSES AND RAPID ANTIBODY PRODUCTION

(71) Applicant: University of Virginia Patent Foundation, Charlottesville, VA (US)

(72) Inventor: Steven L. Zeichner, Bethesda (US)

(21) Appl. No.: 17/769,899

(22) PCT Filed: Oct. 16, 2020
(86) PCT No.: PCT/US2020/055995

§ 371 Date: Apr. 18, 2022

Related U.S. Application Data
(60) Provisional application No. 62/916,873, filed on Oct. 18, 2019.

(51) Int. Cl. A61K 39/00; C12N 1/20; C07K 16/12; A61P 35/00; C12N 15/70

(52) U.S. Cl. CPC A61K 39/0011; C12N 1/20; C07K 16/1203; A61P 35/00; C12N 15/70; C12N 2510/00; C07K 2317/14; A61K 2039/521; A61K 2039/522; A61K 2039/543; A61K 2039/585

(57) ABSTRACT

Provided are modified bacteria and derivatives thereof that express antigens of interest. In some embodiments, the bacterium has a reduced genome and induces an enhanced immune response against the antigen of interest when administered to a subject as compared to an immune response that would have been induced by a bacterium of the same strain that has a full complement of genes. In some embodiments, the antigen is expressed on a surface of a bacterium. Also provided are method for producing antibody against antigens of interest, vaccine compositions, methods for vaccinating subjects, methods for treating cancers in subjects, methods for modulating inappropriate and undesirable immune response, methods for targeting materials in or on a human or animal that may be the cause of disease or otherwise undesirable phenotypes, and expression vectors for expressing antigens on the surface of reduced genome bacteria.

### CROSS REFERENCE TO RELATED APPLICATIONS

This application claims benefit of U.S. provisional application Ser. No. 62/916,873, filed Oct. 18, 2019. The disclosure of this Provisional application is incorporated herein by reference in its entirety.

### TECHNICAL FIELD

The presently disclosed subject matter relates to compositions and methods useful for inducing cellular and humoral immune responses, such as but not limited to compositions and methods employed in the context of vaccine development and antibody production. In representative embodiments, the presently disclosed subject matter relates to bacteria modified to have reduced expression of genes, such as by having a reduction of the bacterial genomes and, in some embodiments, using those bacteria to express antigens of interest. The presently disclosed subject matter also relates in some embodiments to vaccine compositions and materials to elicit useful antibody responses from humans and animals comprising modified bacteria.

### BACKGROUND

The development of new, faster, more effective methods to induce immune responses in subjects and to produce custom antibodies against a protein of interest represents a need in the art. The presently disclosed subject matter addresses these and other needs in the art.

Custom antibodies made against a protein of interest enable and inform much of contemporary biomedical research. ELISAs, flow cytometric analyses, immunoblots, immunohistochemistry, immunopreciptations, immunoaffinity purifications—without these and other antibody-dependent techniques modern biomedical research would not exist. However, the production of custom antibodies is a slow process—much slower than might be expected when compared to the pace of antibody produced in response to a natural infection. It takes three months or more from the time a purified protein antigen linked to a carrier protein becomes available to produce a custom polyclonal custom antibody. It may take another three weeks or more to prepare the immunogen. Some natural infections induce the production of antibodies much faster—in days. Indeed, if effective antibodies took months to develop in response to a severe natural infection, the humoral immune response could not help control an infection.

Effective vaccines exist to prevent and treat some human and animal diseases, but there are no vaccines for other important infections of humans and animals. Some existing vaccines do not provide for the rapid development of an effective immune response, and new infectious agents, both naturally occurring infectious agents and maliciously disseminate agents, continue to emerge. Current approaches to inducing immune responses against cancers are slow, cumbersome and expensive. Many pathologic processes, such as autoimmune and inflammatory diseases result from the dysregulation or inappropriate expression of components of the immune system or inflammatory mediators. Inflammatory and autoimmune diseases can be treated with agents that target inflammatory mediators and components of the immune system. A method to develop more effective vaccines, vaccines that more rapidly induce an immune response against pathogens, cancers, and components of the immune system and mediators of inflammatory processes, and vaccines that be produced quickly in response to new biological threats is urgently needed.

The presently disclosed subject matter addresses these needs in the art and society.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter provides a modified bacterium or derivative thereof. In some embodiments, the modified bacterium has a reduced number of expressed genes and comprises an antigen, optionally an antigen on a surface of a membrane or derivative thereof, wherein the bacterium induces an enhanced immune response against the antigen when administered to a subject as compared to an immune response that would have been induced in the subject by a bacterium of the same strain that has a full complement of expressed genes. In some embodiments, reducing and/or eliminating expression of one or more genes in the bacterium yields the enhanced immunogenicity.

In some embodiments, the modified bacterium is a Gram-negative bacterium, optionally a member of the Enterobacteriaceae. In some embodiments, the bacterium is an Escherichia coli (E. coli).

In some embodiments, the reduced number of expressed genes comprises a reduction of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes. In some embodiments, the reduced number of expressed genes comprises a reduction of expressed genes selected from the group consisting of at least about 2.4%, at least about 15.9%, and at least about 29.7%.

In some embodiments, the antigen is put on the surface of the bacterium by an approach selected from the group consisting of expression by the cell itself, covalent or non-covalent association with the outer membrane, and combinations thereof. In some embodiments, the modified bacterium comprises an autotransporter (AT) expression vector encoding the antigen, wherein the expression on the surface is provided by the AT expression vector. In some embodiments, the autotransporter expression vector comprises a codon optimized sequence encoding the antigen. In some embodiments, the AT expression vector comprises a monomeric autotransporter vector or a trimeric autotransporter vector.

The antigen can be any desired antigen as would be apparent to one of ordinary skill in the art upon a review of the instant disclosure. In some embodiments, the antigen is derived from a microbe. In some embodiments, the antigen is derived from a cancer. In some embodiments the antigen is derived from a host protein that mediates other diseases or undesirable phenotypes, including in some embodiments autoimmune or inflammatory diseases, or diseases in which the expression of a particular host protein mediates a disease process.

In some embodiments, a method for producing an antibody or a desired cell-mediated immune response in a subject is disclosed. In some embodiments, the method comprises providing a modified bacterium in accordance with the presently disclosed subject matter and administering the modified bacterium to a subject in an amount and via a route sufficient to produce an antibody or a desired cell-mediated immune response in the subject against the antigen expressed by the modified bacterium or against cells expressing the antigen. Optionally, the production of the antibody or cell mediated immune response is enhanced in the subject as compared to an immune response produced in a subject by a bacterium of the same strain that has a full complement of expressed genes and that expresses the antigen on its surface. In some embodiments, the administering of the modified bacterium to the subject is intranasally, transmucosally, including but not limited to orally, rectally, and vaginally; subcutaneously, intradermally, intramuscularly, other parenteral routes, or any combination thereof.

In some embodiments, a vaccine composition comprising a modified bacterium according to the presently disclosed subject matter and a pharmaceutically acceptable carrier is provided. Optionally, the vaccine composition further comprises one or more adjuvants. In some embodiments, the modified bacterium is a live attenuated bacterium or a killed whole cell bacterium. In some embodiments, the vaccine composition is adapted to be administered orally, rectally, vaginally, intra-nasally, parenterally, intradermally, subcutaneously, or intramuscularly. In some embodiments, the vaccine composition further comprises an adjuvant.

In some embodiments, a method for vaccinating a subject in need thereof is provided. In some embodiments, the method comprises providing a vaccine composition of the presently disclosed subject matter and administering the vaccine composition to the subject. In some embodiments, a method for treating a cancer or inappropriate immune responses or expression or production of a deleterious material in a subject in need thereof is provided, the method comprising providing a vaccine composition according to the presently disclosed subject matter and administering the vaccine to the subject. In some embodiments, a method for treating a cancer in a subject in need thereof is provided. In some embodiments, the method comprises providing a vaccine composition of the presently disclosed subject matter and administering the vaccine to the subject. In some embodiments, the vaccine composition is administered orally, rectally, vaginally, intra-nasally, parenterally, intradermally, subcutaneously, or intramuscularly. In some embodiments, a method for treating an autoimmune process in a subject in need thereof is provided. In some embodiments, the vaccine composition is administered orally, rectally, vaginally, intra-nasally, parenterally, intradermally, subcutaneously, or intramuscularly. In some embodiments, a method for altering the production or expressing of a pathogenic protein, or modifying or attacking or killing cells mediating disease, in a subject in need thereof is provided. In some embodiments, the vaccine composition is administered orally, rectally, vaginally, intra-nasally, parenterally, intradermally, subcutaneously, or intramuscularly.

In some embodiments, an expression vector comprising a nucleotide sequence encoding an antigen is provided. In some embodiments, the expression vector is configured to express the antigen in a modified bacterium of the presently disclosed subject matter. In some embodiments, the antigen is expressed on the surface of the modified bacterium. In some embodiments, the vector comprises an autotransporter (AT) expression vector. In some embodiments, the vector comprises a codon optimized sequence encoding the antigen. In some embodiments, the AT expression vector comprises a monomeric vector or a trimeric vector. In some embodiments, the nucleotide sequence encoding the antigen is positioned under control of an inducible promoter or a constitutive promoter. In some embodiments, the antigen is expressed as a monomer or as a trimer. In some embodiments, the vector is provided in a pharmaceutically acceptable carrier.

Accordingly, it is an object of the presently disclosed subject matter to provide methods and compositions for eliciting immune responses such as but not limited to for the purpose of creating new and more effective vaccines and for antibody production.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and EXAMPLES.

### BRIEF DESCRIPTIONS OF THE DRAWINGS

FIG. 1 is a graph plotting the number of bacterial proteins with imputed locations on the bacterial surface removed increasing as a function of the percent of the genome deleted in an exemplary E. coli strain of the presently disclosed subject matter, which strains were produced by the Tokyo Metropolitan University Group (TMUG GR).

FIG. 2 is a schematic depicting the design of exemplary Gram-negative surface transporter expression cassette plasmid pRIAIDA (SEQ ID NO:1). The plasmid was synthesized to include the following features: a rhamnose inducible promoter (PrhaBAD), the Gram-negative expression cassette including the N-terminal signal sequence (Signal), an HA immunotag (HA-tag), which serves both as a test antigen for the demonstration of the technology and as a “stuffer” sequence that can be removed and replaced with DNA sequence encoding an immunogen-of-interest for the production of a vaccine or immunogen to elicit useful antibodies, a trypsin cleavage site to evaluate surface expression of the HA immunotag (and any other surface expressed protein cloned into the cloning sites flanking the HA-tag coding sequence (here shown as Bbs I sites), the beta-barrel of the autotransporter (AIDA-I Autotransporter), together with the plasmid origin of replication (ori), and the kanamycin resistance gene with its promoter (PKanR, KanR)

FIG. 3 is a photograph of immunoblots showing surface expression and relative amounts of a surface-expressed HA immunotag expressed by the AIDA-I autotransporter in various reduced genome E. coli. Protein extracts were made from aliquots of E. coli that had not been transformed with an HA immunotag expression cassette, wild type (WT) E. coli that had been transformed with a plasmid with an HA immunotag autotransporter surface expression cassette, and reduced genome (2.4% deleted, 15.8% deleted, and 29.7% deleted) E. coli that had been transformed with a plasmid with an HA immunotag autotransporter surface expression cassette with and without exposure to trypsin. Rhamnose was used to induce expression of the HA immunotag. The extracts were analyzed via immunoblotting using a commercial anti-HA monoclonal antibody. HA bands are seen in the extracts from the E. coli transformed with the plasmid having the HA immunotag surface expression cassette. Treatment of the bacteria with trypsin prior to production of the protein extract severely reduced or eliminated the HA band, indicating that HA was placed on the bacterial surface and that there was minimal detectable HA in the bacterial cytoplasm. DNAK (70 kDa) was used as a lane loading control.

FIGS. 4A and 4B are plots showing binding and immunogenicity of a test antigen expressed on the surface of wild type and genome deleted E. coli. FIG. 4A is a plot showing binding of a commercial anti-HA mAb to wild type and genome deleted (2.4%, 15.9%, 29.7%) E. coli assessed by flow cytometry. GR E. coli harboring a pRIAIDA plasmid were grown in LB with kanamycin and induced with rhamnose. The bacteria were pelleted, washed in PBS, and fixed with formalin, then washed in PBS. Cells were incubated with anti-HA mAb (Invitrogen), with secondary anti-mouse IgG-Alexa 488 (BD Bioscience). Stained cells were measured by flow cytometry using a BD FACSCALIBUR™, and data were analyzed with FlowJo v10 to determine the percentage of GR bacteria stained with anti-HA mAb. FIG. 4B is a plot showing the amount of anti-HA antibody, determined using an ELISA with commercial anti-HA mAb standards, obtained 2 weeks after intranasal immunization, displayed as a function of the amount of genome deleted, before (circles) and after (triangles) immunization, with a non-transformed bacteria negative control (leftmost points). Immunization with the highly GR killed whole cell E. coli expressing the HA immunotag on their surfaces elicited production of substantial anti-HA antibodies only two weeks after immunization.

FIG. 5 is a diagram summarizing an exemplary workflow for the rapid production of a vaccine, for the purposes of producing a new vaccine for prophylactic infectious disease (human or veterinary) use, or therapeutic use, or to immunize animals for the purposes of producing immune responses against an antigen. The figure includes a projected timeline for each of the steps in the procedure, with a cumulative estimated time from concept to testable immunogen of 10-14 days. For the experiments described, and for any widely deployed effort to produce new prophylactic or therapeutic vaccine candidates, or to rapidly produce custom antibodies, an immunogen is selected and its coding sequence is optionally codon-optimized for the E. coli or other bacterial vector being employed. The coding sequence is synthesized and then inserted into pRIAIDA or an analogous vector containing the equivalent functionality, the plasmid is transformed into GR E. coli or other GR bacteria, and a host animal is immunized with the GR E. coli or other GR bacteria. As set forth herein, the immune responses elicited by the GR E. coli are compared with responses elicited by immunization with control wt E. coli.

FIG. 6 is a timeline in which the workflow outlined herein yields a significantly more rapid schedule for the production of custom antibodies, one application of the new technology, than previously available. This schematic contrasts conventional polyclonal antibody production with the proposed accelerated workflow of the presently disclosed subject matter. The entries above the line in FIG. 6 are from the ProMab website (https://www.promab.com/rabbit-polyclonal-antibodies). Other companies show essentially similar calendars. The entries below the line in FIG. 6 outline an exemplary accelerated timeline of the presently disclosed subject matter. Please note that for the exemplary proposed timeline, since antigens are produced by synthetic biology and immediately expressed using the bacterial vector, the time to initially produce an immunogen ready for immunization is significantly shortened. While in data it has been shown that intranasal immunization with GR E. coli can yield Abs 2 weeks after only a single prime, in this schema, to account for the potential to further enhance immune responses an optional boost is included in the procedure. If a boost is not included in the final procedure, the process would be further shortened by an additional 10-14 days, but the timeline projections are intentionally conservative so one or more boosts is in some embodiments included. Please note that the timeline for antibody production for the presently disclosed process is not in itself speculative, but is completely consistent with the humoral immune responses elicited by some natural pathogen infections.

FIG. 7 is a diagram of an exemplary tumor vaccine strategy that employs the compositions and methods of the presently disclosed subject matter. In this representative, non-limiting implementation, selected tumor antigens are expressed in the GR E. coli. The left side of the illustration shows how the technology can be used to immunize a cancer patient using known tumor antigens, that is tumor antigens known to be associated with a particular cancer that a patient has. The right side shows an example of how the technology can be used to offer immunotherapy customized for a particular patient's cancer. In this implementation, the tumor is analyzed to discover tumor neoantigens specific to that patient's tumor, either by RNAseq, as shown in the figure, or through another approach, such as a proteomic analysis. The discovery of patient-specific tumor antigens could either proceed through analysis of bulk tumor, or through analysis of isolated tumor cells, or through specific analysis of cells within tumors, such as through laser capture microdissection. In this implementation, tumor neoantigens are identified, DNA sequences encoding the antigens are designed and synthesized, with or without codon optimization, and in some instances modifications in the coding sequence to enhance immune recognition and/or immunogenicity, or for biasing to a particular type of immune response, then cloned into the expression vector, transformed into the GR E. coli or other GR bacteria. A vaccine is produced from the GR bacteria and administered to the cancer patient. In some implementations, additional immune modulators, such as immune checkpoint inhibitors are administered in conjunction with the tumor antigen vaccine(s).

### DETAILED DESCRIPTION

### I. Representative Embodiments

The presently disclosed subject matter relates to the effects on immunogenicity of expressing immunogens, such as vaccine antigens, in bacteria that have a reduced or eliminated expression of genes. Thus, in some embodiments, the bacterium with fewer expressed genes is more immunogenic. By way of example and not limitation, wholesale reduction of the bacterial genome, by small or large scale deletions, is one way this might be accomplished. Other approaches for decreasing expression of one or more genes are contemplated to fall within the scope of the presently disclosed subject matter, such as but not limited to specific knock outs, targeted inactivations or excisions by any one of several approaches (exemplary, but not exclusively through CRSPR/Cas9, TALENS, ZFNs), knock downs, effects on promoters, conditional mutants and/or inducible mutants (for use in better growing up the bacteria that may be growth restricted by the mutations or gene inactivations, or in live attenuated bacterial vaccines. In some representative, non-limiting embodiments, genes affecting surface structures can affected. Expression of protein structures can affected, as can be non-protein structures.

In accordance with some embodiments of the presently disclosed subject matter, the terms “genome reduced” “genome reduction” or “GR” are used interchangeably and encompasses actual deletions but also other modifications, such as inactivation, functional inactivation, and/or mutation, that reduce expression of one or more genes. In some embodiments, reducing and/or eliminating expression of genes in the bacteria yields the enhanced immunogenicity. In some embodiments, the reduced number of expressed genes comprises a reduction of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes. In some embodiments, the reduced number of expressed genes comprises a reduction of expressed genes selected from the group consisting of at least about 2.4%, at least about 15.9%, and at least about 29.7%.

In some aspects of the presently disclosed subject matter, there is a steady increase in immunogenicity as more and more genes are deleted, without a distinct “threshold effect” or notable discontinuity, which supports that beyond the effects of deleting a specific gene, there are effects due to the overall quantitative reduction in the number of genes.

Genes may be completely or partially deleted, for example by the methods employed by Hashimoto et al., 2005 and by the lambda Red systems described by Datsenko et al., 2000; by CRSPR/Cas9; and other methods to delete, inactivate, or decrease expression of bacteria genes as would be apparent to one of ordinary skill in the art upon a review of the instant disclosure.

The antigen or immunogen is any antigen against which an immune response is desired. One or more such antigens can be provided by the modified bacterium. Representative, non-limiting examples of antigens include an antigen to modulate autoimmune responses, an antigen for which it might be therapeutically useful to produce an immune response, such as fibrosis associated with atherosclerosis or the amyloid plaques of Alzheimer's disease or other degenerative diseases; an antigen used induce an immune response against specific components of the immune system to modify autoimmune or allergic diseases; and/or combinations thereof.

The presently disclosed subject matter provides the following exemplary non-limiting aspects and embodiments.

Bacteria that have a reduced expression of a set of genes and that have an immunogen of interest, such as but not limited to on their surfaces, elicit an enhanced immune response against that immunogen compared to wild type, non-gene reduced bacteria.

In some embodiments, an expression vector comprising a nucleotide sequence encoding an antigen is provided. In some embodiments, the expression vector is configured to express the antigen in a modified bacterium of the presently disclosed subject matter. The presently disclosed subject matter encompasses any suitable expression vector as would be apparent to one of ordinary skill in the art upon a review of the instant disclosure. In some embodiments, the antigen is expressed on the surface of the modified bacterium. In some embodiments, the vector comprises an autotransporter (AT) expression vector. In some embodiments, the vector comprises a codon optimized sequence encoding the antigen. In some embodiments, the AT expression vector comprises a monomeric vector or a trimeric vector. In some embodiments, the nucleotide sequence encoding the antigen is positioned under control of an inducible promoter or a constitutive promoter. In some embodiments, the antigen is expressed as a monomer or as a trimer. In some embodiments, the vector is provided in a pharmaceutically acceptable carrier. Thus, one way the immunogen can be placed on the surface of the bacteria is using an autotransporter (monomeric or trimeric), by itself, or in the context of a foreign protein or scaffold to enhance/improve formation of desired immunogen. The autotransporter expression is not the only way to express antigens. Immunogens can be placed on the surface of the bacteria using other technologies, for example by covalent or non-covalent linkage, absorption, affinity tag, and the like. Using other technologies to place immunogens on the surfaces of the reduced genome bacteria provides for the production of immunogens and/or vaccines directed against proteins or other antigens that cannot be expressed on the bacterial surface using autotransporters or against non-protein antigens (such as but not limited to polysaccharides).

There are many other ways to express antigens and/or to specifically place them on the surfaces of the bacteria, or even inside the bacteria, such as but not limited to covalent coupling of the antigen to the surface of the bacteria, association of the bacteria with antigen non-covalently using an affinity tag, non-specific adsorption, addition of a binding moiety to the antigen followed by mixing the antigen with the bacteria.

The autotransporter expression cassette approach enables a synthetic biology solution: the protein antigen need not be isolated/purified/conjugated to carrier protein. Only the identity of the protein is needed. Then the coding sequence can be rapidly synthesized and cloned into the appropriate expression vector, followed by expression in the GR bacteria.

The wild type/native protein can be used, or a component of the protein can be used, if it is desirable to produce an immune response only against a particular component of the protein. A mutated version of the protein can be used, to enhance immune responses or to bias immune responses (in a non-exclusive example, humoral vs. cellular), or direct immune responses toward a particular mutant version of the gene (for example, in a cancer application).

The antigen or immunogen, used interchangeably herein, can be used to elicit an immune response against a pathogen, as in developing a prophylactic vaccine. The immunogen can be used to elicit an immune response against a pathogen, as in developing a therapeutic vaccine, for example to treat a chronic infectious disease, including chronic viral diseases. One example would be HIV in an HIV-infected patient. TB is another application as are parasitic diseases.

The immunogen can be used to elicit an immune response against a non-pathogen, for example to manipulate the microbiome.

The immunogen can be used to elicit an immune response some other self-protein/proteins, as a way of modifying inflammatory or autoimmune diseases, for example by targeting particular cells or subsets of cells in the patient's immune system.

The immunogen can be used to elicit an immune response against a tumor antigen, as in developing a prophylactic vaccine against particular cancers in which tumor antigens are overexpressed, enhance tumor immune surveillance.

The immunogen can be used to elicit an immune response against a tumor antigen, as in developing a therapeutic tumor vaccine.

The therapeutic tumor vaccine can be directed against known tumor antigens (native or modified for targeting or enhanced immunogenicity). In other words, stocks of premade tumor vaccines against know tumor antigens can be prepared. These could be used singly or in combination. They could be used by themselves, or along with treatments to enhance immune responses against those tumor antigens, such as immune checkpoint inhibitors.

The therapeutic tumor vaccine can be directed against tumor antigens identified on a custom basis. In other words, an individual patient's cancer can be studied, using techniques such as RNAseq, deep sequencing of the tumor DNA, or proteomics approaches and then alone, or in comparison to normal tissues from the same or other patients select and design sequence encoding the tumor antigen (native or modified). These could be used singly or in combination. The vaccines could be used by themselves, or along with treatments to enhance immune responses against those tumor antigens, such as immune checkpoint inhibitors.

All of the above prophylactic and therapeutic uses can be in humans or animals. For example, the technology can be used to make veterinary prophylactic infectious disease vaccines.

The immunogen can be used to elicit the rapid production of antibodies in animals for the purposes of producing antibodies. These can be, for example, custom polyclonal antibodies, obtained directly from various species used to make custom polyclonal antibodies, such as rabbits, goats, sheep, horses, cows, and camelidae. The antibodies can be obtained from serum or from colostrum.

The immunogen can be used to immunize animals (e.g. mice, but also other species, including rabbits) to accelerate the production of monoclonal antibodies, since the first step in making a monoclonal antibody is to immunize an animal so that it makes antibodies, so that its spleen cells can be fused with myeloma cells to make a hybridoma. Such monoclonal antibodies can be used in all the analytic, diagnostic, and therapeutic ways in which monoclonal antibodies are typically used.

The genome reduced bacterial immunogen can be a killed/inactivated bacterium or a live bacterium. The bacteria can be killed/inactivated in many different ways: formalin, glutaraldehyde, heat, radiation, other chemicals. The bacteria can be whole bacteria or derivatives of whole bacteria, for example ghost cells, blebs, vesicles. The vaccine could also be fragments of the genome reduced cells. Such derivatives are prepared in accordance with techniques recognized in the art, as would be apparent to one of ordinary skill in the art up on a review of the instant disclosure.

The bacterium can be any bacterium, including Gram-negative bacteria. E. coli are not the only genome reduced bacteria that can be used. Other Gram-negative bacteria can be used, and other genome reduced strains of other bacteria can be used, such as but not limited to genome reduced Salmonella or even Vibrio. Such genome reduced versions of other bacterial species are prepared in accordance with techniques recognized in the art, as would be apparent to one of ordinary skill in the art up on a review of the instant disclosure, and then use them to express immunogens, such as vaccine antigens. Thus, in some embodiments the bacteria are from Enterobacteriaceae, such as but not limited to Salmonella, Klebsiella, Shigella, Yersinia. In some embodiments, representative bacteria can be chosen via a systematic review of the taxonomic tree: and thus, can include all Proteobacteria.

Reduced genome bacteria, used by themselves, without a recombinant antigen on their surfaces, could be used to elicit useful immune responses against those bacteria. Such reduced genome bacteria could be used as prophylactic or therapeutic vaccines or to manipulate the composition of the microbiome.

Thus, in some aspects and embodiments, the presently disclosed subject matter relates to strategies for the rapid production of better immunogens for the production of new vaccines, including prophylactic vaccines for infectious diseases of humans and animals and therapeutic vaccines for cancer immunotherapy and other disorders where modulation of an immune response is therapeutically helpful. These strategies include:

1. Direct antigen production in a bacterial cell employing a synthetic biology approach in which the antigen of interest is expressed directly in the bacteria, directed by recombinant coding sequence. In some implementations, these proteins are placed on the cell surface, and in some implementations Gram-negative autotransporter protein expression cassettes are used to place the antigen-of-interest on the bacterial cell surface. Instead of purifying the protein antigen and conjugating it to carrier protein, antigen coding sequences can be cloned into an expression cassette. In the Gram-negative autotransporter embodiment discussed herein, these autotransporters (Type 5 Secretion Systems) place the antigen on the cell surface as the vaccine immunogen. This obviates any need to isolate or synthesize the protein antigen, purify the antigen, couple the antigen to an appropriate carrier, and prepare a parental immunization, saving up to several weeks.

2. Use of genome reduced bacteria (such as but not limited to E. coli) to express the antigen. In some embodiments, the bacteria are Gram-negative bacteria, and in some embodiments the Gram-negative bacteria are E. coli. In representative embodiments, surface expressed antigen would be more accessible to the immune system and elicit better immune responses by expressing the antigens, such as but not limited to vaccine antigens, in genome reduced bacteria, in some embodiments on the surfaces of genome reduced bacteria, in some embodiments Gram-negative bacteria, and in one example on the surfaces of genome reduced (GR) E. coli.

3. Intranasal immunization. As a representative, non-limiting route, intranasal immunization exposes M cells and dendritic cells directly to the immunogen, and the oropharyngeal mucosa has a large amount of lymphoid tissue, which produces enhanced immune responses to intranasally administered immunogens. Disclosed herein are data indicating that combining the above strategies can yield an unexpectedly potent induction of antibody against the test antigen. However, the presently disclosed subject matter encompasses any route of administration as would be apparent to one of ordinary skill in the art upon a review of the instant disclosure, including but not limited to topical, oral, rectally, vaginally, intravenous, intramuscular, intra-arterial, intramedullary, intrathecal, intraventricular, transdermal, subcutaneous, intraperitoneal, enteral, sublingual, or in the case of a neoplasm, intratumorally.

4. Exponential increasing (exp-inc) immunization. In a representative, non-limiting embodiment, sequential, rapid exposure to increasing amounts of immunogen can yield enhanced immune responses, thought to occur because such immunogen exposure kinetics mimic the antigen exposure a host would experience in the face of a severe, poorly controlled infection, which would trigger an enhanced immune response. The immunogens discussed herein can be used in exp-inc immunization regimens to enhance immune responses against the antigen. Conversely, the immunogens described herein can be used in exponential decreasing dose administration patterns, or at repeated low doses to elicit a tolerizing response.

Aspects of the presently disclosed subject matter relate at least in part to the use of genome reduced bacteria to produce an antigen capable rapidly inducing an immune response against an antigen. The antigen-expressing genome reduced bacteria enable rapid antibody production for use in making custom polyclonal antibodies and materials needed (for example plasma cells) for monoclonal antibodies. The antigen-expressing genome reduced bacteria also can serve as vaccine immunogens designed to elicit immune responses that protect against infectious agents or vaccine immunogens designed to elicit a therapeutic immune response against cancers or a therapeutic immune response designed to otherwise therapeutically modulate immune responses, for example in treatment autoimmune diseases.

As set forth herein, expressing an antigen in a genome reduced bacterium can yield substantially higher binding of an antibody directed against the antigen to the bacteria and that bacteria expressing the test antigen elicit a significantly higher immune response against the test antigen when an animal is immunized with genome reduced bacteria expressing that test antigen than when immunized with wild type bacteria, and that bacteria with progressively increasing amounts of genome deletion elicited increasingly potent immune responses.

Further, in some aspects, an enhanced immune response in accordance with some aspects of the presently disclosed subject matter involves enhanced cytotoxic T-cell responses directed against tumor cells and/or enhanced antibody responses directed against tumor antigens expressed in or on the surfaces of the tumor cells Enhanced cytotoxic T-cell responses may be directed against previously identified tumor antigens or against newly identified antigens selected based on the analysis of genes or proteins differentially expressed in the cancer. Anti-tumor antibody responses may be directed against antigens conventionally targeted by monoclonal antibodies currently in use for cancer therapeutics or against novel antigens.

### II. ABBREVIATIONS AND ACRONYMS

| | |
|---|---|
| Ab | Antibody |
| AIDA | adhesin involved in diffuse adherence |
| AT | autotransporter (AT) |
| GALT | gut associated lymphoid tissue |
| KWC | killed whole cell |
| Mab | monoclonal antibody |
| OM | outer membrane |
| PC | phosphatidyl choline |
| TCIU | tissue culture infectious units |
| TMD | transmembrane domain |

### III. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

A disease or disorder is “alleviated” if the severity of a symptom of the disease, condition, or disorder, or the frequency with which such a symptom is experienced by a subject, or both, are reduced.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an increased immune response when used in combination with a specific antigen.

As use herein, the terms “administration of” and or “administering” a compound should be understood to mean providing a compound of the presently disclosed subject matter or a prodrug of a compound of the presently disclosed subject matter to a subject in need of treatment.

As used herein, the term “aerosol” refers to suspension in the air. In particular, aerosol refers to the particlization or atomization of a formulation of the presently disclosed subject matter and its suspension in the air.

As used herein, an “analog” of a chemical compound is a compound that, by way of example, resembles another in structure but is not necessarily an isomer (e.g., 5-fluorouracil is an analog of thymine).

As used herein, “amino acids” are represented by the full name thereof, by the three letter code corresponding thereto, or by the one-letter code corresponding thereto, as indicated in the following Table 1:

| | 3- | 1- | |
|---|---|---|---|
| | Letter | Letter | Functionally Equivalent |
| Full Name | Code | Code | Codons |
| Aspartic Acid | Asp | D | GAC GAU |
| Glutamic Acid | Glu | E | GAA GAG |
| Lysine | Lys | K | AAA AAG |
| Arginine | Arg | R | AGA AGG CGA CGC CGG CGU |
| Histidine | His | H | CAC CAU |
| Tyrosine | Tyr | Y | UAC UAU |
| Cysteine | Cys | C | UGC UGU |
| Asparagine | Asn | N | AAC AAU |
| Glutamine | Gln | Q | CAA CAG |
| Serine | Ser | s | ACG AGU UCA UCC UCG UCU |
| Threonine | Thr | T | ACA ACC ACG ACU |
| Glycine | Gly | G | GGA GGC GGG GGU |
| Alanine | Ala | A | GCA GCC GCG GCU |
| Valine | Val | V | GUA GUC GUG GUU |
| Leucine | Leu | L | UUA UUG CUA CUC CUG CUU |
| Isoleucine | Ile | I | AUA AUC AUU |
| Methionine | Met | M | AUG |
| Proline | Pro | P | CCA CCC CCG CCU |
| Phenylalanine | Phe | F | UUC UUU |
| Tryptophan | Trp | W | UGG |

The term “amino acid” is used interchangeably with “amino acid residue,” and may refer to a free amino acid and to an amino acid residue of a peptide. It will be apparent from the context in which the term is used whether it refers to a free amino acid or a residue of a peptide. Amino acids have the following general structure:

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydrooxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as exchanges within one of the following five groups:
I. Small aliphatic, nonpolar or slightly polar residues: Ala, Ser, Thr, Pro, Gly;
IL Polar, negatively charged residues and their amides: Asp, Asn, Glu, Gln;
III. Polar, positively charged residues: His, Arg, Lys;
IV. Large, aliphatic, nonpolar residues: Met Leu, Ile, Val, Cys
V. Large, aromatic residues: Phe, Tyr, Trp

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms including, for example, polyclonal antibodies, monoclonal antibodies, Fv, Fab and F(ab)₂, as well as single chain antibodies and humanized antibodies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates. The term “immunogen” is used interchangeably with “antigen” herein.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this presently disclosed subject matter, and is effective in killing or substantially inhibiting the growth of microbes. “Antimicrobial” as used herein, includes antibacterial, antifungal, and antiviral agents.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the peptides encompasses natural or synthetic portions of a longer peptide or protein that are capable of specific binding to their natural ligand or of performing the desired function of the protein, for example, a fragment of a protein of larger peptide which still contains the epitope of interest and is immunogenic.

The term “biological sample,” as used herein, refers to samples obtained from a subject, including, but not limited to, skin, hair, tissue, blood, plasma, cells, sweat and urine.

As used herein, the term “carrier molecule” refers to any molecule that is chemically conjugated to the antigen of interest that enables an immune response resulting in antibodies specific to the native antigen.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell is a cell being examined.

A “pathoindicative” cell is a cell which, when present in a tissue, is an indication that the animal in which the tissue is located (or from which the tissue was obtained) is afflicted with a disease or disorder.

A “pathogenic” cell is a cell which, when present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

As used herein, a “derivative” of a bacterium, antigen, composition or other compound refers to a bacterium, antigen, composition or other compound that may be produced from bacterium, antigen, composition or other compound of similar structure in one or more steps.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting a risk or propensity to an addictive related disease disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly at least five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCCS' and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993. This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

By the term “immunizing a subject against an antigen” is meant administering to the subject a composition, a protein complex, a DNA encoding a protein complex, an antibody or a DNA encoding an antibody, which elicits an immune response in the subject, and, for example, provides protection to the subject against a disease caused by the antigen or which prevents the function of the antigen.

The term “immunologically active fragments thereof” will generally be understood in the art to refer to a fragment of a polypeptide antigen comprising at least an epitope, which means that the fragment at least comprises 4 contiguous amino acids from the sequence of the polypeptide antigen.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit,” as used herein when referring to a function, refers to the ability of a compound of the presently disclosed subject matter to reduce or impede a described function. In some embodiments, inhibition is by at least 10%, in some embodiments by at least 25%, in some embodiments by at least 50%, and in some embodiments, the function is inhibited by at least 75%. When the term “inhibit” is used more generally, such as “inhibit Factor I”, it refers to inhibiting expression, levels, and activity of Factor I.

The term “inhibit a complex,” as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting, or applying, or administering” includes administration of a compound of the presently disclosed subject matter by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, or rectal approaches.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container which contains the identified compound presently disclosed subject matter or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to, through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels

The term “nasal administration” in all its grammatical forms refers to administration of at least one composition of the presently disclosed subject matter through the nasal mucous membrane to the bloodstream for systemic delivery of at least one compound of the presently disclosed subject matter. The advantages of nasal administration for delivery are that it does not require injection using a syringe and needle, it avoids necrosis that can accompany intramuscular administration of drugs, trans-mucosal administration of a drug is highly amenable to self administration, and intranasal administration of antigens exposes the antigen to a mucosal compartment rich in surrounding lymphoid tissues, which can promote the development of a more potent immune response, particularly more potent mucosal immune responses.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the presently disclosed subject matter.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides but when used in the context of a longer amino acid sequence can also refer to a longer polypeptide.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of contracting the disease and/or developing a pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., 1994).

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this presently disclosed subject matter.

As used herein, “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is in some embodiments at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990b; Altschul et al., 1990a; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when it is in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

By the term “vaccine,” as used herein, is meant a composition which when inoculated into a subject has the effect of stimulating an immune response in the subject, which serves to fully or partially treat and/or protect the subject against a condition, disease or its symptoms. In one aspect, the condition is HIV. TB is another application as are parasitic diseases. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### IV. Compositions and Methods

In some embodiments, a pharmaceutical composition comprising one or more components of the presently disclosed subject matter is administered orally. In one aspect, it is administered intra-nasally, rectally, vaginally, parenterally, employing intradermal, subcutaneously, or intramuscularly. In some embodiments, the pharmaceutical composition is a vaccine.

The system can also be used to express other viral proteins on the surface of bacteria to be used for immunization or treatment directed against the other viral proteins.

The presently disclosed subject matter provides a series of proteins or peptides and systems to produce or express those peptides in the context of cell structures, such as a lipid bilayer and other membrane structures found to have immunogenic activity that can be used singly or in combination to elicit an immunogenic response and are useful for preventing and treating viral infections (such as HIV), and microbial infections (such as TB). The presently disclosed subject matter could also be used to produce immunizing antigens targeting the conserved regions of other virion envelope proteins, for or example, a universal influenza vaccine.

In some embodiments, the presently disclosed subject matter provides a modified bacterium expressing a set of peptides that can be used together as a cocktail or individually as a component of a vaccine (immunogen) to prevent or to treat any condition, disease, and/or disorder as described herein. When administered, the bacterium comprising the cocktail or combination of peptides elicits an immunogenic response. The presently disclosed subject matter further encompasses the use of biologically active homologues of the peptides and wells as biologically active fragments of the peptides. The homologues can, for example, comprise one of more conservative amino acid substitutions, additions, or deletions.

In some embodiments, the presently disclosed subject matter provides an immunogenic vaccine composition for use in treating and preventing viral infections and other microbial infections. In some aspects, the composition comprises at least one isolated peptide selected from the group of peptides disclosed herein, or biologically active fragments or homologs thereof. In some aspects, the immunogenic vaccine composition is a system comprising a viral peptide provided by a bacterium in accordance with the presently disclosed subject matter. The vaccine composition can also include an adjuvant or a pharmaceutically acceptable carrier. In one aspect, at least two peptides are included in the composition. Any combination of the peptides can be used.

In some embodiments, an immunogenic fragment or homolog of a peptide of the presently disclosed subject matter is used. In some embodiments, the biologically active fragments or homologs of the peptide share at least about 50% sequence identity with the peptide. In some aspects, they share at least about 75% sequence identity with the peptide. In yet other aspects, they share at least about 95% sequence identity with the peptide.

In some embodiments, at least one of the active fragments or homologs being used comprises a serine or alanine amino acid substitution for a cysteine residue. In some embodiments, at least one of the active fragments or homologs being used comprises at least one conservative amino acid substitution. The presently disclosed subject matter encompasses the use of amino acid substitutions at any of the positions, as long as the resulting peptide maintains the desired biologic activity of being immunogenic. The presently disclosed subject matter further includes the peptides where amino acids have been deleted or inserted, as long as the resulting peptide maintains the desired biologic activity of being immunogenic.

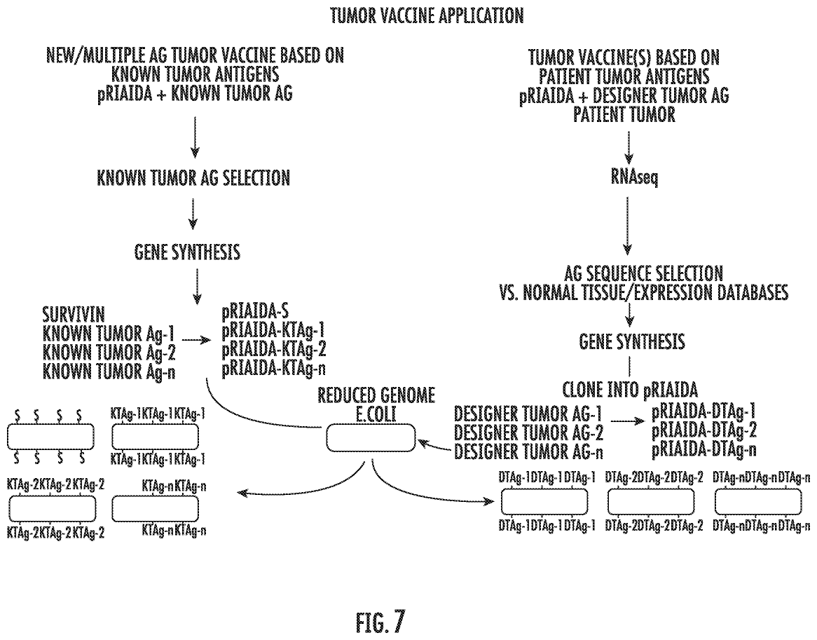

In some embodiments, the methods of the presently disclosed subject matter provide for administering the vaccine composition to a subject at least about 2 times to about 50 times. In some embodiments, the method comprises administering the vaccine composition to a subject at least about 5 times to about 30 times. In some embodiments, the methods of the presently disclosed subject matter provide for administering the vaccine composition to a subject at least about 10 times to about 20 times. The method also provides for administering the composition daily, or weekly, or monthly. One of ordinary skill in the art can design a regimen based on the needs of a subject, taking into account the age, sex, and health of the subject. An exemplary vaccination strategy employing the compositions and methods of the presently disclosed subject matter is provided in FIG. 7.

As described herein, the peptides provided by the modified bacterium are immunogenic, so a useful composition comprising one or more of the peptides of the presently disclosed subject matter, even when using active fragments or homologs, or additionally short peptides, elicits an immunogenic response.

In some aspects, a homolog of a peptide of the presently disclosed subject matter is one with one or more amino acid substitutions, deletions, or additions, and with the sequence identities described herein. In some aspects, the substitution, deletion, or addition is conservative. In some aspects, a serine or an alanine is substituted for a cysteine residue in a peptide of the presently disclosed subject matter.

In some embodiments, the subject is a mammal. In another embodiment, the mammal is a human.

The presently disclosed subject matter encompasses the use of purified isolated, recombinant, and synthetic peptides.

The presently disclosed subject matter further provides methods for producing peptides which are not easily soluble in an aqueous solution, by immediately expressing the peptides on the surface of the bacteria.

The methods and compositions of the presently disclosed subject matter encompass multiple regimens and dosages for administering the peptides of the presently disclosed subject matter for use in preventing and treating diseases and disorders caused by infectious agents. For example, a subject can be administered a combination of peptides, such as a combination of peptides provided by a bacterium, or a combination of bacteria expressing different peptides, of the presently disclosed subject matter once or more than once. The frequency and number of doses can vary based on many parameters, including the age, sex, and health of the subject. In some embodiments, up to 50 doses are administered. In some embodiments, up to 40 doses are administered, and in another up to 30 doses are administered. In some embodiments, up to 20 doses are administered, and in another up to 10 doses are administered. In some embodiments, 5-10 doses are administered. In some embodiments, 5, 6, 7, 8, 9, or 10 doses can be administered.

In some embodiments, bacteria expressing a peptide or bacteria expressing two or more peptides are administered more than once daily, in another daily, in another on alternating days, in another weekly, and in another, monthly. Treatment periods may be for a few days, or about a week, or about several weeks, or for several months. Follow-up administration or boosters can be used as well and the timing of that can be varied.

The amount of bacteria expressing a peptide or derivative of the bacteria administered per dose can vary as well. For example, in some embodiments, the compositions and methods of the presently disclosed subject matter include a range of peptide amounts (for example as provided by bacteria expressing a peptide) between about 1 nanogram of each peptide per dose to about 10 milligrams of immunogen per dose. In some embodiments, the number of micrograms is the same for each peptide. In some embodiments, the number of micrograms is not the same for each peptide. In some embodiments, the range of amounts of each immunogen administered per dose is from about 1 nanogram to about 10 milligrams.

Subjects can be monitored before and after bacteria administration for antibody levels against the immunogens being administered (for example as provided by bacteria expressing a peptide) and by monitoring T cell responses, including CD4⁺ and CD8⁺. Methods for these tests are routinely used in the art and are either described herein or, for example, in publications cited herein.

Although a vaccine composition construct, bacteria, mixture of bacteria, derivatives thereof, or cocktail of peptides or a combination therefor is described herein, when more than one bacterial construct or peptide is administered, each different bacterial construct or peptide can be administered separately. When a vaccine composition is administered more than once to a subject, the dose of each bacterial construct or peptide may vary per administration.

To increase the immunological response, various adjuvants may be used depending on the host species, including but not limited to cholera toxin B subunit, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as cholera toxin B subunit, alum, saponins, nucleic acids, LPS, BCG (bacille Calmette-Guerin) and Corynebacterium parvum.

If peptides are to be placed on the genome reduced bacteria following exogenous production and not by protein synthesis by the bacteria themselves, those peptides for use in the presently disclosed subject matter may be readily prepared by standard, well-established techniques, such as solid-phase peptide synthesis (SPPS) as described by Stewart et al., 1984 and as described by Bodanszky & Bodanszky, 1984. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenyl esters.

Examples of solid phase peptide synthesis methods include the BOC method which utilized tert-butyloxicarbonyl as the α-amino protecting group, and the FMOC method which utilizes 9-fluorenylmethyloxycarbonyl to protect the α-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dichloromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide. Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈-, or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation,” a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Decarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and decarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tartaric, citric, benzoic, cinnamic, mandelic, methanesulfonic, ethanesulfonic, p-toluenesulfonic, salicyclic and the like, to provide a water soluble salt of the peptide is suitable for use in the presently disclosed subject matter, for example a GR bacteria with attached additional immunogens.

The presently disclosed subject matter also provides for homologs of proteins and peptides for use in accordance with the presently disclosed subject matter. Homologs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on protein function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Homologs of such polypeptides include those containing residues other than naturally occurring L-amino acids, e.g., D-amino acids or non-naturally occurring synthetic amino acids. The peptides of the presently disclosed subject matter are not limited to products of any of the specific exemplary processes listed herein.

Substantially pure protein or peptide obtained as described herein may be purified by following known procedures for protein purification, wherein an immunological, enzymatic, or other assay is used to monitor purification at each stage in the procedure. Protein purification methods are well known in the art, and are described, for example in Deutscher et al., 1990.

One of ordinary skill in the art will appreciate that when more than one peptide is used (for example as provided by a bacterium expressing two or more peptides or by different bacteria expressing different peptides or derivative of the bacterium) that they do not necessarily have to be administered in the same pharmaceutical composition at the same time, and that multiple administrations can also be used. When multiple injections are used they can be administered, for example, in a short sequence such as one right after the other or they can be spaced out over predetermined periods of time, such as every 5 minutes, every 10 minutes, every 30 minutes, etc. Of course, administration can also be performed by administering a pharmaceutical comprising all components to be administered, such as a cocktail comprising bacteria expressing a peptide or derivative of the bacteria of the presently disclosed subject matter. It can also be appreciated that a treatment regimen may include more than one round of injections, spaced over time such as weeks or months, and can be altered according to the effectiveness of the treatment on the particular subject being treated.

The presently disclosed subject matter provides multiple methods of using specifically prepared bacteria expressing a peptide or derivative of the bacteria, for example, in fresh or lyophilized liposome, proper routes of administration of the bacteria or derivative thereof, proper doses of the bacteria or derivative thereof, and specific combinations of heterologous immunization including priming in one administration route followed by liposome-mediated antigen boost in a different route to tailor the immune responses in respects of enhancing cell mediated immune response, cytokine secretion, humoral immune response, especially skewing T helper responses to be Th1 or a balanced Th1 and Th2 type. For more detail, see U.S. patent application Ser. No. 11/572,453 (published as U.S. Patent Application Publication No. 2008/0193469 and incorporated herein by reference in its entirety), which claims priority to PCT International Patent Application Serial No. PCT/US2005/026102 (published as PCT International Patent Application Publication No. WO 2006/012539 and incorporated herein by reference in its entirety).

A homolog herein is understood to comprise an immunogenic peptide having in some embodiments at least 70%, in some embodiments at least 80%, in some embodiments at least 90%, in some embodiments at least 95%, in some embodiments at least 98%, and in some embodiments at least 99% amino acid sequence identity with the peptides mentioned above and is still capable of eliciting at least the immune response obtainable thereby. A homolog or analog may herein comprise substitutions, insertions, deletions, additional N- or C-terminal amino acids, and/or additional chemical moieties, such as carbohydrates, to increase stability, solubility, and immunogenicity.

In one embodiment of the presently disclosed subject matter, the present immunogenic polypeptides as defined herein, are glycosylated. Without wishing to be bound by any particular theory, it is hypothesized herein that by glycosylation of these polypeptides the immunogenicity thereof may be increased. Therefore, in one embodiment, the aforementioned immunogenic polypeptide as defined herein before, is glycosylated, having a carbohydrate content varying from 10-80 wt %, based on the total weight of the glycoprotein or glycosylated polypeptide. Said carbohydrate content ranges can be from 15-70 wt %, or from 20-60 wt %. In another embodiment, said glycosylated immunogenic polypeptide comprises a glycosylation pattern that is similar to that of the peptides of the human that is treated. It is hypothesized that this even further increases the immunogenicity of said polypeptide. Thus, in one embodiment, the immunogenic polypeptide comprises a glycosylation pattern that is similar to that of the corresponding glycoprotein.

In one embodiment, the source of a peptide comprises an effective amount of at least one immunogenic peptide selected from the peptides described herein, and immunologically active homologs thereof and fragments thereof, or a nucleic acid sequence encoding said immunogenic peptide.

In one embodiment, the present method of immunization comprises the administration of a source of immunogenically active peptide fragments, said peptide fragments being selected from the peptide fragments and/or homologs thereof as defined herein before.

Peptides may advantageously be chemically synthesized and may optionally be (partially) overlapping and/or may also be ligated to other molecules, peptides, or proteins. Peptides may also be fused to form synthetic proteins, as in Welters et al., 2004. It may also be advantageous to add to the amino- or carboxy-terminus of the peptide chemical moieties or additional (modified or D-) amino acids in order to increase the stability and/or decrease the biodegradability of the peptide. To improve immunogenicity, immuno-stimulating moieties may be attached, e.g. by lipidation or glycosylation. To enhance the solubility of the peptide, addition of charged or polar amino acids may be used, in order to enhance solubility and increase stability in vivo.

For immunization purposes, the aforementioned immunogenic peptides for use with the presently disclosed subject matter may also be fused with proteins, such as, but not limited to, tetanus toxin/toxoid, diphtheria toxin/toxoid or other carrier molecules. The polypeptides according to the presently disclosed subject matter may also be advantageously fused to heatshock proteins, such as recombinant endogenous (murine) gp96 (GRP94) as a carrier for immunodominant peptides as described in (see e.g., Rapp & Kaufmann, 2004; Zugel, 2001), or fusion proteins with Hsp70 (PCT International Patent Application Publication No. WO 1999/54464).

The individual amino acid residues of the present immunogenic (poly)peptides for use with the presently disclosed subject matter can be incorporated in the peptide by a peptide bond or peptide bond mimetic. A peptide bond mimetic of the presently disclosed subject matter includes peptide backbone modifications well known to those skilled in the art. Such modifications include modifications of the amide nitrogen, the alpha carbon, amide carbonyl, complete replacement of the amide bond, extensions, deletions, or backbone cross-links. See generally, Spatola, 1983. Several peptide backbone modifications are known and can be used in the practice of the presently disclosed subject matter.

Amino acid mimetics may also be incorporated in the polypeptides. An “amino acid mimetic” as used here is a moiety other than a naturally occurring amino acid that conformationally and functionally serves as a substitute for an amino acid in a polypeptide of the presently disclosed subject matter. Such a moiety serves as a substitute for an amino acid residue if it does not interfere with the ability of the peptide to elicit an immune response. Amino acid mimetics may include non-protein amino acids. A number of suitable amino acid mimetics are known to the skilled artisan, they include cyclohexylalanine, 3-cyclohexylpropionic acid, L-adamantyl alanine, adamantylacetic acid and the like. Peptide mimetics suitable for peptides of the presently disclosed subject matter are discussed by Morgan & Gainor, 1989.

In some embodiments, the present method comprises the administration of a composition (e.g., bacteria or derivative thereof) comprising one or more of the present immunogenic peptides as defined herein above, and at least one excipient. Excipients are well known in the art of pharmacy and may for instance be found in textbooks such as Remington's Pharmaceutical Sciences, 18th ed. (1990).

The present method for immunization may further comprise the administration, and in one aspect, the co-administration, of at least one adjuvant. Adjuvants may comprise any adjuvant known in the art of vaccination or composition for eliciting an immune response and may be selected using textbooks like Colligan et al., eds1994-2004.

Adjuvants are herein intended to include any substance or compound that, when used, in combination with an antigen, to immunize a human or an animal, stimulates the immune system, thereby provoking, enhancing or facilitating the immune response against the antigen, preferably without generating a specific immune response to the adjuvant itself. In one aspect, adjuvants can enhance the immune response against a given antigen by at least a factor of 1.5, 2, 2.5, 5, 10, or 20, as compared to the immune response generated against the antigen under the same conditions but in the absence of the adjuvant. Tests for determining the statistical average enhancement of the immune response against a given antigen as produced by an adjuvant in a group of animals or humans over a corresponding control group are available in the art. The adjuvant preferably is capable of enhancing the immune response against at least two different antigens. The adjuvant of the presently disclosed subject matter will usually be a compound that is foreign to a human, thereby excluding immunostimulatory compounds that are endogenous to humans, such as e.g. interleukins, interferons, and other hormones.

A number of adjuvants are well known to one of ordinary skill in the art. Suitable adjuvants include, e.g., incomplete Freund's adjuvant, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxy-phosphoryloxy)-ethylamine (CGP 19835A, referred to as MTP-PE), DDA (2 dimethyldioctadecylammonium bromide), polylC, Poly-A-poly-U, RIBI™ GERBU™, PAM3™, CARBOPOL™, SPECOL™, TITERMAX™, tetanus toxoid, diphtheria toxoid, meningococcal outer membrane proteins, cholera toxin B subunit, diphtheria protein CRM197. Preferred adjuvants comprise a ligand that is recognized by a Toll-like-receptor (TLR) present on antigen presenting cells. Various ligands recognized by TLR's are known in the art and include e.g. lipopeptides (see e.g., PCT International Patent Application Publication No. WO 2004/110486), lipopolysaccharides, peptidoglycans, lipoteichoic acids, lipoarabinomannans, lipoproteins (from mycoplasma or spirochetes), double-stranded RNA (poly I:C), unmethylated DNA, flagellin, CpG-containing DNA, and imidazoquinolines, as well derivatives of these ligands having chemical modifications.

In some embodiments of the present methods, one or more bacteria expressing a peptide or derivative of the bacteria are typically administered at a dosage of about 1 ug/kg patient body weight or more at least once. Often dosages are greater than 10 μg/kg. According to the presently disclosed subject matter, the dosages range in some embodiments from 1 μg/kg to 1 mg/kg.

In some embodiments typical dosage regimens comprise administering a dosage of in some embodiments 1-1000 ug/kg, in some embodiments 10-500 μg/kg, in some embodiments 10-150 μg/kg, once, twice, or three times a week for a period of one, two, three, four or five weeks. According to some embodiments, 10-100 μg/kg is administered once a week for a period of one or two weeks.

The presently disclosed methods, in some aspects, comprise administration of bacteria expressing a peptide or derivative of the bacteria and compositions comprising them via the injection, transdermal, intranasal, or oral route. In some aspects of the presently disclosed subject matter, the present method comprises vaginal or rectal administration of the present bacteria expressing a peptide or derivative of the bacteria and compositions comprising them.

Another aspect of the presently disclosed subject matter relates to a pharmaceutical preparation comprising as the active ingredient the present source of a polypeptide as defined herein before. More particularly pharmaceutical preparation comprises as the active ingredient one or more of the aforementioned immunogenic peptides, homologues thereof and fragments of said peptides and homologs thereof, as provided by a bacterium expressing a peptide or derivative of the bacterium as defined herein above.

The presently disclosed subject matter further provides a pharmaceutical preparation comprising one or more bacteria expressing a peptide or derivative of the bacteria of the presently disclosed subject matter. The concentration of said peptides in the pharmaceutical composition can vary widely, i.e., from less than about 0.1% by weight, usually being at least about 1% by weight to as much as 20% by weight or more.

The composition may comprise a pharmaceutically acceptable carrier in addition to the active ingredient. The pharmaceutical carrier can be any compatible, non-toxic substance suitable to deliver the immunogenic peptide or bacteria expressing a peptide or derivative of the bacteria to the patient. For polypeptides, sterile water, alcohol, fats, waxes, and inert solids may be used as the carrier. Pharmaceutically acceptable adjuvants, buffering agents, dispersing agents, and the like, may also be incorporated into the pharmaceutical compositions.

In some embodiments, the present bacteria expressing a peptide or derivative of the bacteria are administered by injection. The parenteral route for administration is in accordance with known methods, e.g., injection or infusion by intravenous, intraperitoneal, intramuscular, intra-arterial, subcutaneous, rectal, vaginal, or intralesional routes. The bacteria expressing a peptide or derivative of the bacteria may be administered continuously by infusion or by bolus injection. In some embodiments, a composition for intravenous infusion could be made up to contain 10 to 50 ml of sterile 0.9% NaCl or 5% glucose optionally supplemented with a 20% albumin solution and in some embodiments between 10 μg and 50 mg, in some embodiments between 50 μg and 10 mg, of the bacteria expressing a peptide or derivative of the bacteria. A typical pharmaceutical composition for intramuscular injection would be made up to contain, for example, 1-10 ml of sterile buffered water and in some embodiments between 10 μg and 50 mg, in some embodiments between 50 ug and 10 mg, of the bacteria expressing a peptide or derivative of the bacteria of the presently disclosed subject matter. Methods for preparing parenterally administrable compositions are well known in the art and described in more detail in various sources, including, for example, Remington's Pharmaceutical Sciences, 18th ed., 1990, incorporated by reference in its entirety for all purposes).

For convenience, immune responses are often described in the presently disclosed subject matter as being either “primary” or “secondary” immune responses. A primary immune response, which is also described as a “protective” immune response, refers to an immune response produced in an individual as a result of some initial exposure (e.g., the initial “immunization”) to a particular antigen. Such an immunization can occur, for example, as the result of some natural exposure to the antigen (for example, from initial infection by some pathogen that exhibits or presents the antigen). Alternatively, the immunization can occur because of vaccinating the individual with a vaccine containing the antigen. For example, the vaccine can be a vaccine comprising one or more antigenic epitopes or fragments of the peptides of the presently disclosed subject matter.

In certain embodiments, the disclosed methods and compositions may involve preparing peptides with one or more substituted amino acid residues. In various embodiments, the structural, physical and/or therapeutic characteristics of peptide sequences may be optimized by replacing one or more amino acid residues.

In some embodiments, the presently disclosed subject matter encompasses the substitution of a serine or an alanine residue for a cysteine residue in a peptide of the presently disclosed subject matter. Support for this includes what is known in the art. For example, see the following citation for justification of such a serine or alanine substitution: Kittlesen et al., 1998.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art. For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C₁-C₁₀ carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopropionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or senile, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see e.g., Chou & Fasman, 1974; Chou & Fasman, 1978; Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

The presently disclosed subject matter is also directed to methods of administering the compounds of the presently disclosed subject matter to a subject.

Pharmaceutical compositions comprising the present compositions are administered to an individual in need thereof by any number of routes including, but not limited to, topical, oral, rectally, vaginally, intravenous, intramuscular, intra-arterial, intramedullary, intrathecal, intraventricular, transdermal, subcutaneous, intraperitoneal, intranasal, enteral, topical, sublingual, or rectal means.

The presently disclosed subject matter is also directed to pharmaceutical compositions comprising the bacteria of the presently disclosed subject matter. More particularly, such compounds can be formulated as pharmaceutical compositions using standard pharmaceutically acceptable carriers, fillers, solubilizing agents and stabilizers known to those skilled in the art.

The presently disclosed subject matter also encompasses the use of pharmaceutical compositions of an appropriate compound, homolog, fragment, analog, or derivative thereof to practice the methods of the presently disclosed subject matter, the composition comprising at least one appropriate compound, homolog, fragment, analog, or derivative thereof and a pharmaceutically-acceptable carrier.

The pharmaceutical compositions useful for practicing the presently disclosed subject matter may be administered to deliver a dose of between 1 ng/kg/day and 100 mg/kg/day. Pharmaceutical compositions that are useful in the methods of the presently disclosed subject matter may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the presently disclosed subject matter.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the presently disclosed subject matter may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the presently disclosed subject matter may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology. A formulation of a pharmaceutical composition of the presently disclosed subject matter suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the presently disclosed subject matter may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the presently disclosed subject matter may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the presently disclosed subject matter may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the presently disclosed subject matter may also be prepared, packaged, or sold in a formulation suitable for rectal administration, vaginal administration, parenteral administration

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the presently disclosed subject matter may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder dispensing container such as a device comprising the active ingredient dissolved or suspended in a low-boiling propellant in a sealed container.

In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the presently disclosed subject matter formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the presently disclosed subject matter.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the presently disclosed subject matter may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Remington's Pharmaceutical Sciences, 18th ed., 1990, which is incorporated herein by reference.

Typically, dosages of the composition of the presently disclosed subject matter which may be administered to an animal, preferably a human, range in amount from 1 μg to about 100 g per kilogram of body weight of the subject. While the precise dosage administered will vary depending upon any number of factors, including but not limited to, the type of animal and type of disease state being treated, the age of the animal and the route of administration. In one embodiment, the dosage of the compound will vary from about 10 μg to about 10 g per kilogram of body weight of the animal. In another embodiment, the dosage will vary from about 10 mg to about 1 g per kilogram of body weight of the subject.

The composition may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the sex and age of the subject, etc.

The presently disclosed subject matter further provides kits comprising bacteria expressing a peptide or derivative of the bacteria of the presently disclosed subject matter useful for eliciting an immunogenic response, and further includes an applicator and an instructional material for the use thereof.

### V. Other Embodiments

In some embodiments, the presently disclosed subject matter also provides other systems by which antigens and/or immunogens of interest can be expressed in, on the surface of, or otherwise by bacteria. Thus, it is understood that the autotransporter expression system described herein is not the only way to express antigens. There are many other ways to express antigens and to specifically place them on the surfaces of the bacteria, or even inside the bacteria.

Similarly, the presently disclosed subject matter also provides modified bacteria other than modified E. coli. By way of example and not limitation, other Gram-negative bacteria can also be employed, and other genome reduced strains of other bacteria could also be used. Examples of other bacteria that could be employed include genome reduced Salmonella or Vibrio. As such, one of ordinary skill in the art could employ the present disclosure as a guide to construct genome reduced versions of other bacterial species for use to express vaccine antigens.

In some embodiments of the presently disclosed subject matter, the modified bacteria can be inactivated. Various methods and approaches for inactivating bacteria for use in immunizations are known to those of skill in the art, and include without limitation use of formalin and/or glutaraldehyde.

Additionally, after review of the instant disclosure one of ordinary skill in the art would also recognize that the purpose of the modified bacterium is primarily to provide a structure in which to provide the immunogen of interest to the immune system of the subject to be immunized. As such, the modified bacterium need not be a fully functional bacterium capable of living, reproducing, etc. As such, in addition to modifications that reduce the genomes of the bacteria, other bacterial derivatives can also be employed. Such derivatives include, but are not limited to ghost cells, bacterial fragments of cells, including but not limited to isolated outer membrane fragments, blebs, etc.

Furthermore, whereas in some embodiments the presently disclosed subject matter relates to the rapid production of antibodies, the presently disclosed subject matter also relates in some embodiments to the production of prophylactic vaccines for infectious diseases and/or therapeutic vaccines for infectious diseases (such as but not limited to chronic infectious diseases like HIV, other chronic viral diseases, TB, and/or parasitic diseases), therapeutic vaccines for cancer (e.g., off the shelf vaccines directed at know cancer antigens) and custom vaccines designed based on the analysis of the cancer neoantigens for a given patient's cancer (i.e., a personalized anti-cancer vaccine), and therapeutic vaccines for other diseases, particularly diseases involving inflammatory processes, like autoimmune diseases, fibrosis, atherosclerosis, etc.

The antigen can be any desired antigen as would be apparent to one of ordinary skill in the art upon a review of the instant disclosure. In some embodiments, the antigen is derived from a microbe. In some embodiments, the antigen is derived from a cancer. In some embodiments the antigen is derived from a host protein that mediates other diseases or undesirable phenotypes, including in some embodiments autoimmune or inflammatory diseases, or diseases in which the expression of a particular host protein mediates a disease process. In some embodiments, the antigen is derived from a cancer, or a target of an inappropriate or undesirable immune response, or a component of the host immune system, such as (but not exclusively), a host immune system component that, when targeted for destruction or inactivation or activation, alter an undesirable immune response.

In some embodiments, a method for producing an antibody or a desired cell-mediated immune response in a subject is disclosed. In some embodiments, the method comprises providing a modified bacterium in accordance with the presently disclosed subject matter and administering the modified bacterium to a subject in an amount and via a route sufficient to produce an antibody or a desired cell-mediated immune response in the subject against the antigen expressed by the modified bacterium or against cells expressing the antigen. Optionally, the production of the antibody or cell mediated immune response is enhanced in the subject as compared to an immune response produced in a subject by a bacterium of the same strain that has a full complement of expressed genes and that expresses the antigen on its surface. In some embodiments, the administering of the modified bacterium to the subject is intranasally, transmucosally, including but not limited to orally, rectally, and vaginally; subcutaneously, intradermally, intramuscularly, other parenteral routes, or any combination thereof.

In some embodiments, a method for vaccinating a subject in need thereof is provided. In some embodiments, the method comprises providing a vaccine composition of the presently disclosed subject matter and administering the vaccine composition to the subject. In some embodiments, a method for treating a cancer or inappropriate immune responses or expression or production of a deleterious material in a subject in need thereof is provided, the method comprising providing a vaccine composition according to the presently disclosed subject matter and administering the vaccine to the subject. In some embodiments, a method for treating a cancer in a subject in need thereof is provided. In some embodiments, the inappropriate immune response or expression or production of a deleterious material is an autoimmune process, a method for altering the production or expressing of a pathogenic protein, and/or modifying or attacking or killing cells mediating disease. In some embodiments, the vaccine composition is administered orally, rectally, vaginally, intra-nasally, parenterally, intradermally, subcutaneously, or intramuscularly.

In some embodiments, the presently disclosed subject matter provides cancer antigens to immunize the endogenous immune system, i.e., “vaccinating” the subject against their own cancer. In some embodiments, the cancer is a drug resistant cancer or drug sensitive cancer. In some embodiments, the cancer is a cancer characterized by the presence of or as a solid tumor or liquid tumor, or is a cancer of hematologic origin. Thus, in some embodiments, the cancer is selected from the group comprising, but not limited to, pancreatic cancer, breast cancer, prostate cancer, lung cancer, head and neck cancer, non-Hodgkin's lymphoma, acute myelogenous leukemia, acute lymphoblastic leukemia, neuroblastoma, and glioblastoma.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Example 1

Construction of an Exemplary Antigen Expression Plasmid

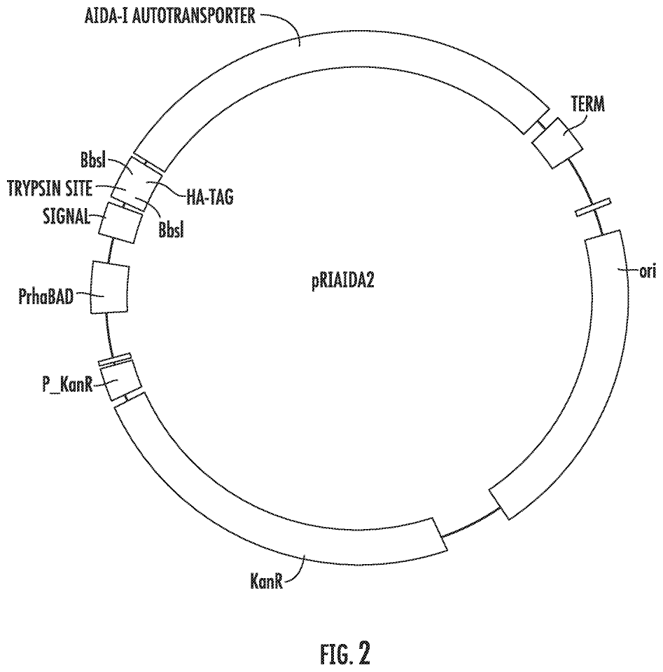

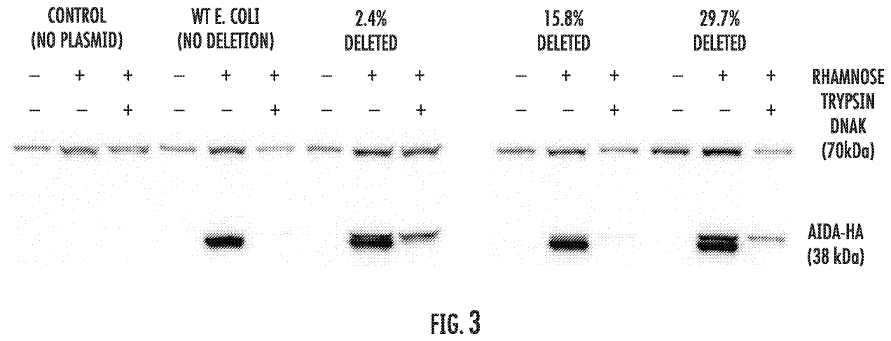

As an exemplary, non-limiting implementation, a Gram-negative AT recombinant expression system for rapid Ab production and immunization was constructed. In experiments, as an exemplary, non-limiting implementation that can used for the expression of antigen in bacteria, plasmid pRIAIDA (SEQ ID NO: 1), which has a rhamnose inducible AIDA-I Gram-negative AT expression cassette for expression optimization, with a cloning site that enables DNA encoding an antigen of interest to be expressed using the inducible AT expression cassette so that bacteria express the encoded protein on their surfaces, and flanked by a trypsin site was placed in the coding sequence to evaluate surface expression of antigens. In experiments, a sequence encoding a widely-used influenza virus HA immunotag (YPYDVPDYA; SEQ ID NO: 2) was inserted into the surface expression cassette to produce plasmid pRIAIDA-HA. FIG. 2 shows the map of the pRIAIDA-HA plasmid. A trypsination experiment confirming that that HA immunotag was present on the exterior of bacteria when expressed in an AIDA-I expression cassette that includes a trypsin site in the coding sequence was performed, and the results are shown in FIG. 3.

### Example 2

Evaluation of Antibody Binding to Gr E. Coli Expressing a Test Immunogen and the Ability of Gr E. Coli Expressing a Test Immunogen on its Surface to Elicit an Immune Response

While only a representative approach, intranasal immunization has a number of advantages as a route of administration. Intranasal immunization leads to the direct exposure in the nasal mucosa of M cells and dendritic cells to the immunogen. In addition, there are abundant nasopharyngeal lymphoid tissues with large numbers of other antigen presenting cells, like macrophages, and many T cells and B cells. Intranasal immunization can also induce potent tissue-resident effector and effector memory CD8+ T cell immunity, and can also be particularly effective in producing antibodies in other body compartments if, for example, secretory mucosal antibodies are useful. In addition, since most pathogens enter the host across mucosal surfaces, if a productive way to rapidly elicit potent antibody response is to mimic a significant pathogen threat, mucosal immunization might be expected to elicit better immune responses than a more traditional parenteral route.

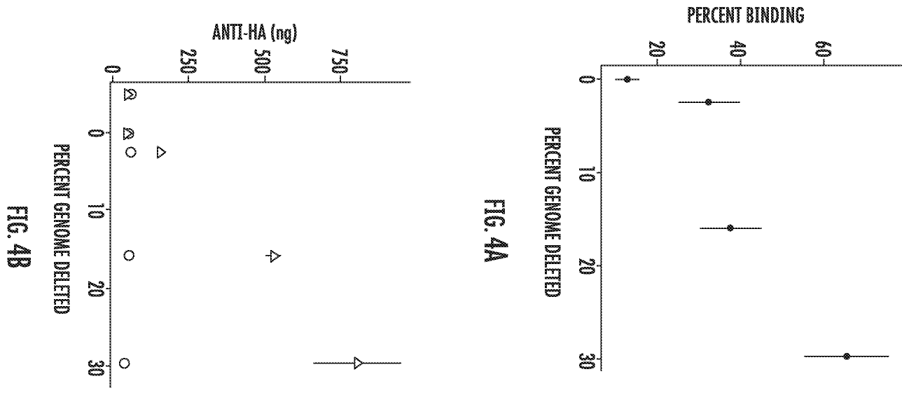

As a test of the new method for rapidly producing highly immunogenic vaccines, made by expressing an antigen in genome reduced bacteria, pRIAIDA-HA was transformed into wild type parental E. coli and also into three GR E. coli. Binding of a commercial anti-HA mAb to the bacteria was evaluated via flow cytometry (FIG. 4A). The ability of the bacteria to bind the anti-HA mAb was found to be increased significantly as the fraction of the genome deleted increased.

This data shows that when an antigen is expressed in a GR bacteria, and more particularly in a Gram-negative bacteria, and more particularly on the surface of a GR bacteria that it is much more accessible to antibody binding, which is a useful surrogate for the immune system as whole. The greater ability to bind antibody and interact with the immune system translates into a better ability elicit a potent immune response directed against that antigen. This is particularly true since many other components of the bacteria, such as LPS, pili, and fimbriae are pathogen-associated molecular patterns, recognized by Toll-like receptors, which should enhance immune responses to recombinant immunogens expressed in the bacteria. The data also indicate that in the wild type bacteria, antigens expressed on the bacterial surface are in some way hidden or “cloaked” from the immune system, and so less able to elicit an immune response. This also suggests that removing large numbers of proteins from the surfaces of bacteria enhance immune responses against all non-removed proteins present on the bacterial surface, including native bacterial proteins, which is useful in making vaccines against bacterial pathogens.

### Example 3

Evaluation of the Ability of Gr E. Coli Expressing a Test Immunogen on its Surface to Elicit an Immune Response

As disclosed herein, expressing an immunogen in a genome reduced bacterium greatly increased the recognition of that antigen by the immune system and elicited anti-immunogen antibodies. That finding suggested that immunogens expressed in genome reduced bacteria would be more accessible to and better recognized by the immune system in general, which implied that expressing an immunogen in a genome reduced bacteria could yield a substantially enhanced immune response against that immunogen. This substantially enhanced immune response against an antigen of interest can then be exploited in this system to: 1) Make new and better prophylactic and therapeutic vaccines for infectious diseases, by expressing pathogen antigens capable of being targeting by an inactivating or neutralizing immune response, 2) Make new and better therapeutic vaccines for cancer targeting tumor specific antigens, 3) Modulate the immune system to clear and/or attack or inactive molecules or structures mediating the pathogenesis of disease, including autoimmune or inflammatory diseases or diseases mediated by the overproduction or overexpression of particular molecules, and 4) Rapid production of custom polyclonal and/or monoclonal antibodies useful for analytic, therapeutic, and industrial purposes. To establish whether expressing an antigen of interest in a genome reduced bacteria would yield an enhanced immune response, vaccines were prepared from wild type and genome reduced bacteria having different degrees of genome reduction. The bacteria were transformed with the pRIADA plasmid that expresses the HA immunotag as a test vaccine antigen. Expression of the HA immunotag was induced with rhamnose and expression of the HA immunotag on the surface of the bacteria was verified by flow cytometry (FIG. 4A).

Mice were immunized intranasally with 10⁸ formalin-fixed wild type and genome reduced bacteria expressing the HA immunotag. After 2 weeks, blood was collected from the immunized mice and the sera tested using an ELISA with a commercial anti-HA mAb as a standard. The quantity of anti-HA antibodies in the mouse sera was determined using the ELISA in pre-immune sera (circles) and after immunization (triangles). It was determined that intranasal immunization with GR E. coli expressing the test immunogen on their surfaces elicited the production of large amounts of Abs in only 2 weeks, and that the ability of the bacteria expressing the test antigen to elicit production of the Ab increased substantially in bacteria with more genes deleted. These data were confirmed using immuno-dot blots that indicated that the same amount of HA was produced per cell regardless of extent of the genome reduction, which would tend to support the hypothesis that the increased binding and immunogenicity was due to increased antigen accessibility in the GR strains and/or an absence of immunoinhibitory surface structures in the GR strains. While in this representative instance the immunoinhibitory components are on the surface of the bacterium, in other instances the immunoinhibitory components can be elsewhere on the bacteria. The examples then show that expressing a well-established test antigen on the surface of genome reduced bacteria leads to substantially increased binding to those bacteria of a monoclonal antibody that recognizes the test antigen and that immunizing an animal with genome reduced bacteria expressing a well-established test antigen on its surface elicits a much more potent immune response than immunizing animals with wild type bacteria expressing the same antigen.

### Example 4

Strategies for Producing Genome Reduced (Gr) Bacteria

The production and characterization of custom antibodies made against a protein of interest is a slow process that can take several months from the time a purified protein antigen linked to a carrier protein becomes available to produce a custom polyclonal custom antibody. It can take a further several weeks or more to prepare the immunogen. The development of a new, faster method to produce custom antibodies against a protein of interest would have great benefit for essentially all biomedical research.

Representative Approach: Immunization with killed whole cell GR E. coli presented intranasally via exp-inc immunization schedules will dramatically decrease the time needed to produced effective custom antibodies.

This EXAMPLE relates to optimizing genome reduced bacterial immunization for rapid antibody production in mice and to demonstrating rapid production of polyclonal antibodies in rabbits using optimized immunization procedures.

The contributions of the modifications to immunization described above on immune responses directed against a test antigen are systematically tested. With an optimized new immunization protocol in hand, the ability of that protocol to elicit production of immune responses against a test antigen in rabbits is confirmed, comparing the new procedures to conventional immunization procedures. A significant reduction in the time to produce custom antibodies would greatly aid progress in essentially all areas of biomedical research.

Gram-negative autotransporters. Gram-Autotransporter (AT) (also termed Autodisplay or Type 5 Secretion System) proteins are a protein family that mediates protein placement into Gram-bacterial outer membranes, with one region anchored in the membrane lipid bilayer and another exposed to the extracellular environment [references 13-16]. AT proteins have 3 key domains: An N-terminal signal sequence that directs protein across the inner membrane via a secA mechanism, a C-terminal β-barrel that inserts into the Gram-OM, yielding a pore-like structure, and a central passenger protein domain that transits through the β-barrel pore to be exposed extracellularly, attached to the β-barrel, which remains anchored in the OM. Native passenger protein coding sequence can be replaced with sequence encoding another protein, yielding a recombinant AT protein. The AT thus ‘displays’ recombinant passenger protein to the extracellular environment, anchored in and closely adjacent to the OM lipid bilayer. About 2×10⁵ recombinant proteins can be placed on each cell's surface [reference 16]. This non-limiting representative approach can be employed in some aspects of the presently disclosed subject matter.

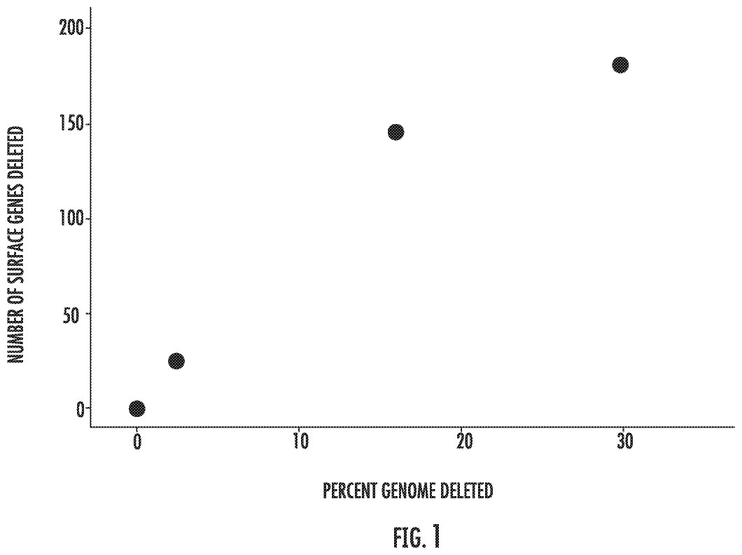

Genome reduced E. coli. To better understand how the number of gene products on the surface of the E. coli strains changed in the TMUG GR strains, the names of the genes in each deletion (LD5510, LD5119, and LD5125) from the National BioResource Project E. coli Strain website created by the National Institute of Genetics, Japan (https://shigen.nig.acjp/ecoli/strain/resource/longDeletion/lddTableInfo) were compiled, with additional gene information from EcoCyc (https://ecocyc.org/), and UniProt (https://www.uniprot.org/), including gene name, protein name, location, function, gene ontology, and other notes about the gene. The information was organized into tables and sorted by deletion. FIG. 1 shows how the number of bacterial gene products with an imputed location on the exterior of the cell were eliminated in the GR E. coli. A large number of surface gene products are eliminated in the GR E. coli strains.

An exemplary list of genes that can be deleted from E. coli is presented in Table 2.

TABLE 2

| | Deleted in | Deleted in | Deleted in | | | | | |
|---|---|---|---|---|---|---|---|---|
| Gene | LD5510 | LD5119 | LD5125 | Outer | Integral to | | | Cell |
| Name | (2.4%) | (15.9%) | (29.7%) | Membrane | Membrane | Membrane | Pilus | Surface |
| acrZ | 0 | 0 | 1 | 1 | 0 | 0 | 0 | 0 |
| actP | 0 | 1 | 1 | 0 | 1 | 0 | 0 | 0 |
| adiC | 0 | 1 | 1 | 0 | 1 | 0 | 0 | 0 |
| aer | 0 | 1 | 1 | 0 | 1 | 0 | 0 | 0 |
| alsC | 0 | 1 | 1 | 0 | 1 | 0 | 0 | 0 |
| alx | 0 | 1 | 1 | 0 | 1 | 1 | 0 | 0 |
| amiD | 0 | 0 | 1 | 1 | 0 | 0 | 0 | 0 |
| arsB | 0 | 1 | 1 | 0 | 1 | 0 | 0 | 0 |
| arti | 0 | 0 | 1 | 0 | 0 | 1 | 0 | 0 |
| artJ | 0 | 0 | 1 | 0 | 0 | 1 | 0 | 0 |
| basS | 0 | 1 | 1 | 0 | 1 | 0 | 0 | 0 |
| bcsB | 0 | 1 | 1 | 0 | 1 | 1 | 0 | 0 |
| bcsC | 0 | 1 | 1 | 1 | 0 | 0 | 0 | 0 |
| cadB | 0 | 1 | 1 | 0 | 1 | 0 | 0 | 0 |
| clsC | 0 | 1 | 1 | 0 | 0 | 1 | 0 | 0 |
| copA | 0 | 1 | 1 | 0 | 1 | 0 | 0 | 0 |
| esgA | 0 | 1 | 1 | 0 | 0 | 0 | 1 | 0 |
| csgB | 0 | 1 | 1 | 1 | 0 | 0 | 1 | 0 |
| esgE | 0 | 1 | 1 | 1 | 0 | 0 | 0 | 0 |
| csgF | 0 | 1 | 1 | 1 | 0 | 0 | 0 | 0 |
| csgG | 0 | 1 | 1 | 1 | 0 | 0 | 0 | 0 |
| dctA | 0 | 1 | 1 | 0 | 1 | 0 | 0 | 0 |
| dcuB | 0 | 1 | 1 | 0 | 1 | 0 | 0 | 0 |
| dcuS | 0 | 1 | 1 | 0 | 1 | 1 | 0 | 0 |
| dinQ | 0 | 1 | 1 | 0 | 1 | 1 | 0 | 0 |
| dppB | 0 | 1 | 1 | 0 | 1 | 0 | 0 | 0 |
| dppC | 0 | 1 | 1 | 0 | 1 | 0 | 0 | 0 |
| dtpB | 0 | 1 | 1 | 0 | 1 | 0 | 0 | 0 |
| ecpA | 0 | 0 | 1 | 0 | 0 | 0 | 1 | 0 |
| ecpC | 0 | 0 | 1 | 1 | 0 | 0 | 0 | 0 |
| ecpD | 0 | 0 | 1 | 0 | 0 | 0 | 1 | 0 |
| eptA | 0 | 1 | 1 | 0 | 1 | 1 | 0 | 0 |
| eptB | 0 | 1 | 1 | 0 | 1 | 1 | 0 | 0 |
| exuT | 0 | 1 | 1 | 0 | 1 | 1 | 0 | 0 |
| fdnG | 0 | 0 | 1 | 0 | 0 | 1 | 0 | 0 |
| fdrA | 0 | 1 | 1 | 0 | 1 | 0 | 0 | 0 |
| fecA | 1 | 1 | 1 | 0 | 0 | 1 | 0 | 0 |
| fecE | 1 | 1 | 1 | 0 | 0 | 1 | 0 | 0 |
| fimA | 1 | 1 | 1 | 1 | 0 | 0 | 0 | 0 |
| fimD | 1 | 1 | 1 | 1 | 0 | 0 | 0 | 0 |
| fimF | 1 | 1 | 1 | 1 | 0 | 0 | 0 | 0 |
| fimG | 1 | 1 | 1 | 1 | 0 | 0 | 0 | 0 |
| fimH | 1 | 1 | 1 | 1 | 0 | 0 | 0 | 0 |
| fimi | 1 | 1 | 1 | 0 | 0 | 0 | 1 | 0 |
| fiu | 0 | 0 | 1 | 1 | 0 | 0 | 0 | 0 |
| fsr | 0 | 1 | 1 | 0 | 1 | 0 | 0 | 0 |
| gadB | 0 | 0 | 1 | 0 | 0 | 1 | 0 | 0 |
| gfcD | 0 | 1 | 1 | 1 | 0 | 0 | 0 | 0 |
| gfcE | 0 | 1 | 1 | 1 | 0 | 0 | 0 | 0 |
| glnH | 0 | 0 | 1 | 0 | 0 | 1 | 0 | 0 |
| gltP | 0 | 1 | 1 | 0 | 1 | 0 | 0 | 0 |
| gltP | 0 | 1 | 1 | 0 | 1 | 0 | 0 | 0 |
| gntP | 1 | 1 | 1 | 0 | 1 | 0 | 0 | 0 |
| gutQ | 0 | 0 | 1 | 0 | 0 | 1 | 0 | 0 |
| hdeD | 0 | 1 | 1 | 0 | 1 | 1 | 0 | 0 |
| hsU | 0 | 0 | 1 | 0 | 0 | 1 | 0 | 0 |
| insH1 | 0 | 1 | 1 | 1 | 0 | 0 | 0 | 0 |
| mdtF | 0 | 1 | 1 | 1 | 0 | 0 | 0 | 0 |
| mdtM | 1 | 1 | 1 | 0 | 1 | 0 | 0 | 0 |
| mdtN | 0 | 1 | 1 | 0 | 1 | 0 | 0 | 0 |
| mdtN | 0 | 1 | 1 | 0 | 1 | 0 | 0 | 0 |
| mdtO | 0 | 1 | 1 | 0 | 1 | 1 | 0 | 0 |
| mdtO | 0 | 1 | 1 | 0 | 1 | 1 | 0 | 0 |
| mdtP | 0 | 1 | 1 | 1 | 0 | 0 | 0 | 0 |
| mdtQ | 0 | 1 | 1 | 1 | 0 | 0 | 0 | 0 |
| melB | 0 | 1 | 1 | 0 | 1 | 0 | 0 | 0 |
| mltB | 0 | 0 | 1 | 1 | 0 | 0 | 0 | 0 |
| nanC | 1 | 1 | 1 | 1 | 1 | 0 | 0 | 0 |
| nrfD | 0 | 1 | 1 | 0 | 1 | 0 | 0 | 0 |
| nrfE | 0 | 1 | 1 | 0 | 1 | 0 | 0 | 0 |
| nrfE | 0 | 1 | 1 | 0 | 1 | 0 | 0 | 0 |
| ompC | 0 | 0 | 1 | 1 | 0 | 0 | 0 | 0 |
| ompG | 0 | 0 | 1 | 1 | 0 | 0 | 0 | 0 |
| ompN | 0 | 0 | 1 | 1 | 0 | 0 | 0 | 0 |
| ompX | 0 | 0 | 1 | 1 | 0 | 0 | 0 | 0 |
| opgB | 1 | 1 | 1 | 0 | 0 | 1 | 0 | 0 |
| pgaA | 0 | 1 | 1 | 1 | 0 | 0 | 0 | 0 |
| pgaB | 0 | 1 | 1 | 1 | 0 | 0 | 0 | 0 |
| phnC | 0 | 1 | 1 | 0 | 0 | 1 | 0 | 0 |
| phnE | 0 | 1 | 1 | 0 | 1 | 1 | 0 | 0 |
| pitA | 0 | 1 | 1 | 0 | 1 | 0 | 0 | 0 |
| proP | 0 | 1 | 1 | 0 | 1 | 0 | 0 | 0 |
| qmcA | 0 | 1 | 1 | 0 | 1 | 1 | 0 | 0 |
| rhsA | 0 | 1 | 1 | 0 | 1 | 1 | 0 | 0 |
| rhsB | 0 | 1 | 1 | 0 | 1 | 1 | 0 | 0 |
| rhsD | 0 | 1 | 1 | 0 | 1 | 1 | 0 | 0 |
| rzoR | 0 | 0 | 1 | 1 | 0 | 0 | 0 | 0 |
| sip | 0 | 1 | 1 | 1 | 0 | 0 | 0 | 0 |
| sstT | 0 | 1 | 1 | 0 | 1 | 1 | 0 | 0 |
| tdcC | 0 | 1 | 1 | 0 | 1 | 0 | 0 | 0 |
| Tsr | 1 | 1 | 1 | 0 | 0 | 1 | 0 | 0 |
| uspB | 0 | 1 | 1 | 0 | 1 | 0 | 0 | 0 |
| wecH | 0 | 1 | 1 | 0 | 1 | 1 | 0 | 0 |
| wza | 0 | 1 | 1 | 1 | 0 | 0 | 0 | 0 |
| xylH | 0 | 1 | 1 | 0 | 1 | 0 | 0 | 0 |
| yaiO | 0 | 0 | 1 | 1 | 0 | 0 | 0 | 0 |
| ybaL | 0 | 1 | 1 | 0 | 1 | 0 | 0 | 0 |
| ybaT | 0 | 1 | 1 | 0 | 1 | 0 | 0 | 0 |
| ybbD | 0 | 1 | 1 | 0 | 1 | 1 | 0 | 0 |
| ybbJ | 0 | 1 | 1 | 0 | 1 | 0 | 0 | 0 |
| ybbM | 0 | 1 | 1 | 0 | 1 | 0 | 0 | 0 |
| ybbP | 0 | 1 | 1 | 0 | 1 | 1 | 0 | 0 |
| ybbW | 0 | 1 | 1 | 0 | 1 | 0 | 0 | 0 |
| ybbY | 0 | 1 | 1 | 0 | 1 | 1 | 0 | 0 |
| ybhC | 0 | 0 | 1 | 1 | 0 | 0 | 0 | 0 |
| ybjC | 0 | 0 | 1 | 0 | 0 | 1 | 0 | 0 |
| yceK | 0 | 1 | 1 | 1 | 0 | 0 | 0 | 0 |
| yddB | 0 | 0 | 1 | 1 | 0 | 0 | 0 | 0 |
| yddL | 0 | 0 | 1 | 1 | 0 | 0 | 0 | 0 |
| ydeQ | 0 | 0 | 1 | 0 | 0 | 0 | 1 | 0 |
| ydeR | 0 | 0 | 1 | 0 | 0 | 0 | 1 | 0 |
| ydeS | 0 | 0 | 1 | 0 | 0 | 0 | 1 | 0 |
| ydeT | 0 | 0 | 1 | 1 | 0 | 0 | 0 | 0 |
| yehA | 0 | 1 | 1 | 0 | 0 | 0 | 1 | 0 |
| yehB | 0 | 1 | 1 | 1 | 0 | 0 | 0 | 0 |
| yehD | 0 | 1 | 1 | 0 | 0 | 0 | 1 | 0 |
| yehR | 0 | 1 | 1 | 0 | 0 | 1 | 0 | 0 |
| yehU | 0 | 1 | 1 | 0 | 1 | 1 | 0 | 0 |
| yehW | 0 | 1 | 1 | 0 | 1 | 0 | 0 | 0 |
| yehY | 0 | 1 | 1 | 0 | 1 | 0 | 0 | 0 |
| yejO | 0 | 0 | 1 | 1 | 0 | 0 | 0 | 0 |
| yfaZ | 0 | 0 | 1 | 1 | 0 | 0 | 0 | 0 |
| vfjV | 0 | 1 | 1 | 0 | 1 | 0 | 0 | 0 |
| ygil | 0 | 1 | 1 | 0 | 1 | 1 | 0 | 0 |
| ygjQ | 0 | 1 | 1 | 0 | 1 | 1 | 0 | 0 |
| ygjR | 1 | 1 | 1 | 0 | 0 | 0 | 0 | 1 |
| ygiV | 0 | 1 | 1 | 0 | 1 | 1 | 0 | 0 |
| yhaH | 0 | 1 | 1 | 0 | 1 | 1 | 0 | 0 |
| yhal | 0 | 1 | 1 | 0 | 1 | 1 | 0 | 0 |
| yhaO | 0 | 1 | 1 | 0 | 1 | 0 | 0 | 0 |
| yhhJ | 0 | 1 | 1 | 0 | 1 | 0 | 0 | 0 |
| yhiD | 0 | 1 | 1 | 0 | 1 | 1 | 0 | 0 |
| yhiM | 0 | 1 | 1 | 0 | 1 | 1 | 0 | 0 |
| yhjD | 0 | 1 | 1 | 0 | 1 | 1 | 0 | 0 |
| yhjE | 0 | 1 | 1 | 0 | 1 | 0 | 0 | 0 |
| yhiG | 0 | 1 | 1 | 0 | 1 | 1 | 0 | 0 |
| yhjK | 0 | 1 | 1 | 0 | 0 | 1 | 0 | 0 |
| yhjV | 0 | 1 | 1 | 0 | 1 | 0 | 0 | 0 |
| yhjX | 0 | 1 | 1 | 0 | 1 | 0 | 0 | 0 |
| yhjY | 0 | 1 | 1 | 0 | 0 | 1 | 0 | 0 |
| yiaA | 0 | 1 | 1 | 0 | 1 | 1 | 0 | 0 |
| yiaB | 0 | 1 | 1 | 0 | 1 | 1 | 0 | 0 |
| yiaD | 0 | 1 | 1 | 0 | 1 | 0 | 0 | 0 |
| yiaM | 0 | 1 | 1 | 0 | 1 | 1 | 0 | 0 |
| yiaN | 0 | 1 | 1 | 0 | 1 | 0 | 0 | 0 |
| yiaT | 0 | 1 | 1 | 1 | 0 | 0 | 0 | 0 |
| yiaV | 0 | 1 | 1 | 0 | 1 | 0 | 0 | 0 |
| yiaW | 0 | 1 | 1 | 0 | 1 | 0 | 0 | 0 |
| yibH | 0 | 1 | 1 | 0 | 1 | 0 | 0 | 0 |
| yibl | 0 | 1 | 1 | 0 | 0 | 1 | 0 | 0 |
| vicE | 0 | 1 | 1 | 0 | 1 | 0 | 0 | 0 |
| yjcH | 0 | 1 | 1 | 0 | 1 | 1 | 0 | 0 |
| yjdF | 0 | 1 | 1 | 0 | 1 | 1 | 0 | 0 |
| yjdL | 0 | 1 | 1 | 0 | 1 | 0 | 0 | 0 |
| yjdO | 0 | 1 | 1 | 0 | 1 | 1 | 0 | 0 |
| yjhE | 1 | 1 | 1 | 0 | 0 | 1 | 0 | 0 |
| yjhP | 1 | 1 | 1 | 0 | 1 | 0 | 0 | 0 |
| yjiG | 1 | 1 | 1 | 0 | 1 | 0 | 0 | 0 |
| yjiH | 1 | 1 | 1 | 0 | 1 | 0 | 0 | 0 |
| yjiJ | 1 | 1 | 1 | 0 | 1 | 0 | 0 | 0 |
| yjiK | 1 | 1 | 1 | 1 | 1 | 0 | 0 | 0 |
| yjiN | 1 | 1 | 1 | 0 | 1 | 0 | 0 | 0 |
| yjiN | 1 | 1 | 1 | 0 | 0 | 1 | 0 | 0 |
| yjiY | 1 | 1 | 1 | 0 | 0 | 1 | 0 | 0 |
| yijL | 1 | 1 | 1 | 0 | 0 | 1 | 0 | 0 |
| ylil | 0 | 0 | 1 | 1 | 0 | 0 | 0 | 0 |
| ynbC | 0 | 0 | 1 | 0 | 0 | 1 | 0 | 0 |
| ynbC | 0 | 0 | 1 | 0 | 0 | 1 | 0 | 0 |
| ynbC | 0 | 0 | 1 | 0 | 0 | 1 | 0 | 0 |
| yneD | 0 | 0 | 1 | 1 | 0 | 0 | 0 | 0 |
| yohC | 0 | 1 | 1 | 0 | 1 | 0 | 0 | 0 |
| yohO | 0 | 1 | 1 | 0 | 1 | 1 | 0 | 0 |
| ypjA | 0 | 1 | 1 | 1 | 0 | 0 | 0 | 0 |
| yqjA | 0 | 1 | 1 | 0 | 1 | 0 | 0 | 0 |
| yqjE | 0 | 1 | 1 | 0 | 1 | 1 | 0 | 0 |
| yqjF | 0 | 1 | 1 | 0 | 1 | 1 | 0 | 0 |
| yqil | 0 | 1 | 1 | 0 | 0 | 0 | 1 | 0 |
| ytcA | 0 | 1 | 1 | 0 | 1 | 1 | 0 | 0 |
| ytcA | 0 | 1 | 1 | 0 | 1 | 1 | 0 | 0 |
| ythA | 1 | 1 | 1 | 0 | 0 | 1 | 0 | 0 |

*Genes are listed by locus name, with the presence (=1) or absence (=0) for each deleted mutant strain and for each location in the bacteria.

Production of Gram-AT recombinant expression systems for rapid Ab production immunizations. In experiments, a plasmid, pRIAIDA (SEQ ID NO:1) (see FIG. 2), which has a rhamnose inducible AIDA-I Gram-AT expression cassette for expression optimization, with a cloning site, flanked by a trypsin site to evaluate surface expression was constructed. In initial experiments, a nucleic acid sequence encoding a widely-used influenza virus HA tag (YPYDVPDYA, SEQ ID NO: 2) was inserted into the surface expression cassette to make pRIAIDA-HA. FIG. 2 shows the map of the plasmid (FIG. 2) and a trypsination experiment confirming that that HA immunotag resides on the exterior of the bacteria when expressed in an AIDA-I expression cassette that includes a trypsin site in the coding sequence (FIG. 3).

Intranasal immunization. Intranasal immunization has a number of significant advantages [references 21-26]. Intranasal immunization leads to the direct exposure in the nasal mucosa of M cells and dendritic cells to the immunogen. In addition, there are abundant nasopharyngeal lymphoid tissues with large numbers of other antigen presenting cells, like macrophages, and many T cells and B cells. Intranasal immunization can induce potent tissue-resident effector and effector memory CD8+ T cell immunity [reference 27]. Intranasal immunization may also be particularly effective in producing antibodies in other body compartments if, for example, secretory mucosal antibodies may be useful [reference 28]. In addition, since most pathogens enter the host across mucosal surfaces, if the hypothesis that the best way to rapidly elicit potent antibody response is to mimic a significant pathogen threat, mucosal immunization might be expected to elicit better immune responses than a more traditional parenteral route.

Evaluation of antibody binding to the GR E. coli expressing a test immunogen on their surfaces and the ability of the GR E. coli expressing a test immunogen on their surfaces to elicit immune responses. As an initial step to test the hypothesis that a new method employing a combination of strategies to enhance immunogenicity for the production of custom Abs, pRIAIDA-HA was transformed into wt parental E. coli and three GR E. coli from the TMUG collection. Binding of a commercial anti-HA mAb to the bacteria was confirmed via flow cytometry (FIG. 4A). It was determined that the ability of the bacteria to bind the anti-HA mAb increased significantly as the fraction of the genome deleted increased. The ability of the GR E. coli to elicit an immune response was tested. Mice were immunized intranasally with 10⁸ formalin fixed bacteria. After 2 weeks, blood was collected from the immunized mice and the sera tested using an ELISA, with commercial anti-HA mAb as a standard. It was found that intranasal immunization with GR E. coli expressing the test immunogen on their surfaces could elicit the production of large amounts of Abs in only 2 weeks, and that the ability of the bacteria expressing the test antigen to elicit production of the Ab increased substantially in bacteria with more genes deleted. Data using immuno-dot blots indicated that the same amount of HA was produced per cell regardless of extent of the genome reduction, which supported the hypothesis that the increased binding and immunogenicity was due to either increased antigen accessibility in the GR strains, or an absence of immunoinhibitory surface structures or both.

Exponentially increasing (exp-inc) immunization schedules. Recent studies comparing alternative immunization schedules have shown that repeated immunizations with exp-inc amounts of immunogens can yield a dramatically improved immune response, with a >10-fold increase in Ab concentrations [reference 34]. The hypothesized reason for the enhanced response is that the exp-inc antigen dosing schedule creates signals to the host immune system similar to those initiated by serious infections that threaten the host, with the enhanced response promoted by prolonged, greater amounts of antigen present in lymph nodes acting to improve antibody maturation. Exp-inc immunization has also been observed to induce more Tfh cells and germinal center B-cells. This non-limiting representative approach can be employed in some aspects of the presently disclosed subject matter.

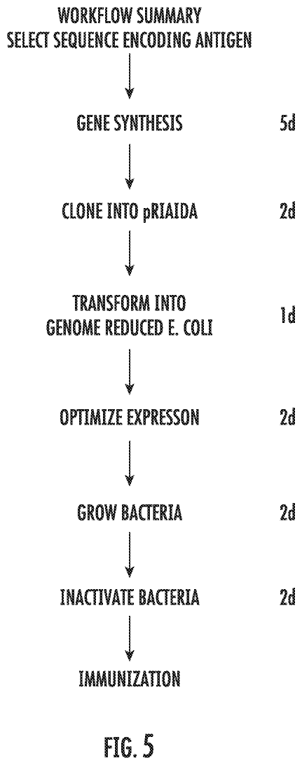

Scientific Premise. Given the known ability of the immune system to respond to threatening pathogens by rapidly producing a potent humoral immune response, it is possible to develop new methods that enable the rapid production of useful polyclonal Abs that can be employed in wide range of biomedical research projects. The data supporting the premise are described above, since it has been demonstrated that a substantial antibody response against a test antigen can be produced in only two weeks. This EXAMPLE is aimed at extending and optimizing the work, and demonstrating that the presently disclosed procedures can produce a rapid, useful polyclonal Ab response in rabbits, the typical species employed in producing useful amounts of polyclonal Abs for biomedical research. Table 3 summarizes the innovations that are employed in the work, along with the different rationales for their use. FIG. 5 contrasts the timeline for the production of custom polyclonal Abs from a leading custom Ab vendor (ProMab) with the timeline for custom Ab production employing the presently disclosed subject matter.

The data showed that the production of antigen via gene synthesis in a Gram-AT expression vector in GR E. coli with intranasal immunization can elicit a strong Ab response in 2 weeks.

TABLE 3
SUMMARY OF FEATURES EMPLOYED AND PROPOSED IN THE NEW RAPID AB
PRODUCTION METHOD

| Feature | Comments |
|---|---|
| Antigen production using synthetic | Rapid immunogen production; Increased immunogencity |
| biology | through production of tandem multimeric antigens; |
| | Increased immunogenicity through amino acid sequence |
| | modifications, Inherent fusion to carrier protein |
| Surface expression via autotransporter | Direct interaction with B-Cell receptors plus |
| (Type 5 secretion system) | processing/presentation by APCs; Auto-adjuvanting |
| expression cassette | through presence of multiple PAMPs on bacteria |
| Genome reduced E. coli | Fewer bacterial proteins interfering with vaccine antigen- |
| | immune system interactions; enhanced exposure of the |
| | immunizing antigen; loss of components masking |
| | bacterial surface antigens from the immune system |
| Intranasal immunization | Direct access to mucosal compartment rich in dendritic |
| | cells and APCs; Needle-free immunization: Induction of |
| | mucosal immunity |
| Exponential increasing immunogen | Mimics signals to the immune system that the |
| dose | immunogen comes from a serious pathogen threat |

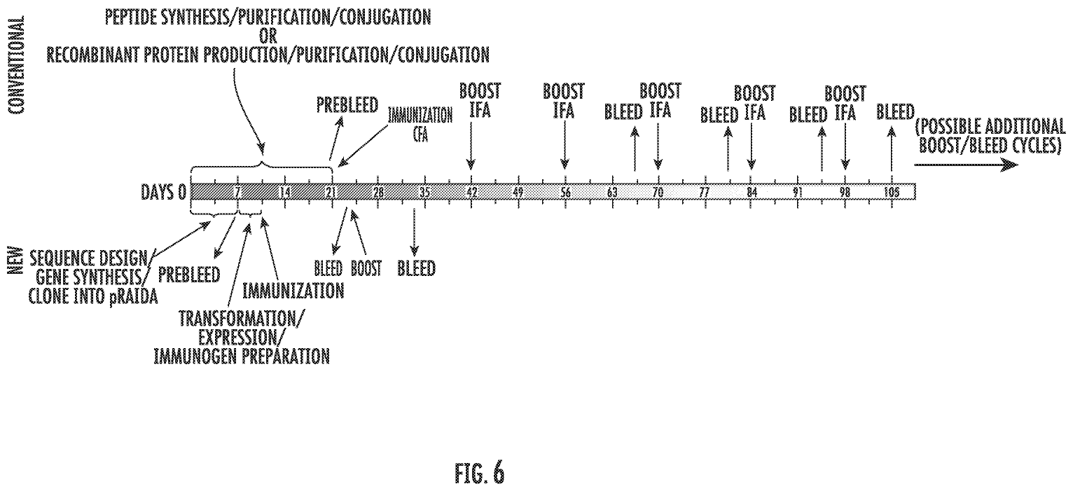

Overview. FIG. 6 summarizes the overall workflow for the presently disclosed subject matter to rapidly produce custom polyclonal Abs. It also indicates areas of additional optimization that are pursued to further enhance the ability of the new procedure to rapidly produce custom polyclonal Abs. Please also note that even though the data presented herein strongly supports that intranasal immunization with GR E. coli expresses an antigen-of-interest on the its surfaces using a Gram-AT expression cassette, additional research can be performed to enable effective, rapid production of custom Abs. This work includes defining the kinetics of the Ab response beyond 2 weeks, a careful analysis of the Ab subtypes to maximize the utility of the custom Abs produced using the new procedure, and work to determine whether induction of the Ab response could be further enhanced. To show that the presently disclosed subject matter is broadly useful for biomedical research, that the procedure can elicit production of custom Abs in a species large enough to make useful quantities of custom Abs, such as rabbit, is also demonstrated.

Optimize Genome Reduced Bacterial Immunization for Rapid Antibody Production in Mice. A series of methods are tested to further optimize rapid production of Abs in mice, then test the most effective of those methods in rabbits, confirming the utility of the presently disclosed subject matter to rapidly produce custom Abs useful for research.

Design of expression cassettes, surface expression and bacterial toxicity testing. An E. coli codon-optimized version of the HA immunotag coding sequence was cloned into pRIAIDA (see FIG. 2), transforming the derivative, into wt non-deleted and GR (2.4%, 15.9%, 29.7%) E. coli [references 19, 20], verifying expression by flow cytometry and surface expression by immunoblot with and without trypsin treatment (see FIG. 3). Since HA is short, highly immunogenic immunotag, a second, larger immunotag is also used to verify that the proposed new Ab production procedure is effective. The ability of the procedure to elicit production of Abs against another model immunogen is also tested. For this second immunogen GFP (MW 27 kDa) is chosen, which is useful for confirming surface expression on the bacteria with IF and flow cytometry. GFP has also been used as a test immunogen in many studies, including studies that showed that E. coli-derived outer membrane vesicles engineered to contain GFP elicited better immune responses than GFP alone [reference 29]. In addition, excellent, well-tested reagents, both recombinant protein and mAbs, are commercially available for GFP (e.g Abcam, SigmaAldrich, ThermoFisher). The AIDA-I AT has been shown to transport GFP and GFP fusions to bacterial surfaces [reference 35]. For the GFP (and any other alternate immunogen constructs), optimum rhamnose induction of expression is determined, following bacterial growth by OD₆₀₀ to determine the maximum expression possible without compromising bacterial replication. The number of immunogen molecules on the surface of each bacteria is determined using immunoblots on bacterial extracts and immunogen protein standards. A goal is at least about 2×10⁵ molecules/bacteria, which has been achieved with other immunogens, and which has been achieved with other antigens, including HA. GFP was also chosen as a widely used marker in biological research that has no clinical use.

Production of immunogens—whole GR E. coli. The methods are those that yielded the data presented herein. Bacteria are grown in LB broth with optimized rhamnose induction, monitoring growth by OD₆₀₀. Bacteria are collected by centrifugation, washed in HBSS, without Ca²⁺/Mg²⁺, 10% formalin is added to a final 0.2% concentration, and incubated at 37° C. for 1 hour with shaking. Aliquots are stored at −80° C. in PBS/10% glycerol, confirming immunogen expression by flow cytometry on thawed stocks. Surface expression is confirmed using a trypsinization procedure, as shown in FIG. 3, either with immunoblots, or flow cytometry (e.g. for GFP).

Evaluating immunogenicity—humoral immunity. A sandwich ELISA was constructed by binding commercial anti-HA mAb (Invitrogen) to blocked, streptavidin-coated strips (Pierce), followed by incubation with commercially produced HA peptide, followed by commercial anti-HA mAb (Invitrogen) with HRP-conjugated goat anti-mouse secondary antibody, assayed using the Tropix CSPD luminescence system, the method used in the results shown in FIGS. 4A and 4B. In those experiments, it was found that anti-HA Abs could be detected to the 1 ng/ml level, below the physiologic level of antibodies against vaccine antigens following vaccination [reference 40].

For the ELISAs on the sera from the immunized animals, essentially the same sandwich ELISA used in FIG. 4B is employed. To develop assays, commercially available HA and GFP proteins were employed as standards. IgG subclasses (IgG1, IgG2a, IgG (Total)), IgA, and IgM are quantitated with HRP-conjugated anti-mouse class and subclass mAbs, with the 1-Step slow TMB-ELISA substrate, or as an alternative, the Tropix CSPD luminescence system, although it is not believed that the added sensitivity of the chemiluminescent assay is required.

Evaluating immunogenicity—cell-mediated immunity. While the presently disclosed subject matter relates to an improved method to rapidly produce custom Abs, it may be helpful in evaluating, comparing, and optimizing the different procedures to have information on the cell mediated immune responses elicited by the different immunization procedures. To characterize the cell-mediated immune responses, ELISpot assays are performed according to the kit manufacturer's instructions (Mabtech). Spleen cells (5×10⁶ cells/mL) obtained at the conclusion of the experiment are plated and stimulated in the presence (or not) of HA (or GFP peptide mix) and incubated for 24 hours. Plates are washed and incubated with biotinylated detection antibody, then incubated with streptavidin-ALP, followed by substrate solution (BCIP/NBT-plus).

In addition to ELIspot assays, assessment of antigen specific T-cell frequency, proliferative capacity, cell surface immunophenotyping, and intracellular cytokine production profiles are determined by flow cytometry to characterize T cell responses. Cells [reference 41] challenged with immunogens are evaluated with a polychromatic (12 color) flow cytometric panel to determine frequencies of HA positive cells, and characterize T_{EM} or T_{CM} phenotypes, since T_{CM} cells vs. T_{EM} cells [references 42-44], which might imply the induction of longer-term immunity, and/or might be helpful for additional boosting and distinguishing between different immunization strategies. For defining T_{EM} and T_{CM} cells, various markers recommended by the Human Immunology Project [reference 45] are employed including CD3, CD4, CD8, CCR7, CD45RA. To assess functionality of the response, intracellular cytokine production and proliferative capacity are measured, and intracellular cytokine staining for IL2, IFN-γ, and TNF-α is performed. Proliferation is evaluated by intracellular staining for Ki-67, which is expressed in cells in S, G₂, and M phases, but not G₀ or G₁ [reference 46]. Fixable amine reactive viability dye is used to eliminate evaluation of dead cells. Non-T cells are identified with a cocktail including anti-CD19, CD14, CD16, CD56, CD11c and CD11b Abs. At least 100,000 events are acquired on a BD 4 laser 17 color FORTESSA™ flow cytometer. Data are analyzed using FlowJo10 (Treestar) software with this gating strategy: 1) Gate for single cells using a FSC area vs. FSC height plot, 2) Gate for live T cells using a CD3 vs. Viability/dump channel (lineage cocktail) plot, 3) Gate for antigen specific CD8+ T cells using CD8 vs. Pentamer plot, 4) Phenotype T_{EM} and T_{CM} using CCR7 vs. CD45RA plot, and 5) Evaluate intracellular cytokine profiles and Ki-67 positivity within the T_{EM} and T_{CM} cell populations.

Immunizations—immunization methods. Immediately before immunization, KWC preparations are thawed on ice and wash in PBS. For the single (non exp-inc) intranasal immunizations, 6 week old mice (CB6F1/J, Jackson) mice are immunized intranasally with 10⁸ cells in 50 ul PBS, boosting at 2 weeks.

Later, immunizations done at a single time with 10⁸ cells are compared with an exponential dose escalation, using doses of 10⁸, 3×10⁸, and 10⁹ cells on alternating days.

For the experiments with the GR E. coli, since the presently disclosed subject matter relates to an accelerated immunization schedule, a rapid schedule with an initial prime and a boost after 2 weeks, as outlined in FIG. 5, is employed. Sera is collected at baseline, before immunization, 2 weeks after immunization, before the boost, and 2 weeks after the boost, at which time the mice are euthanized and larger blood volumes are collected by terminal bleed, along with spleens for the isolation of spleen mononuclear cells for the assays for cell mediated immunity (see below).

As controls (see below for more details on the head-to-head comparisons), mice are immunized SC with the same doses of bacteria used in the intranasal experiments, and with commercially purchased recombinant immunogen protein (HA, GFP), using an initial immunization with protein immunogen emulsified in CFA, followed by boosts in IFA, a standard immunization schedule, to elicit anti-protein humoral immune responses [reference 47]. The schedule is based on that shown in FIG. 5, a typical schedule for the production of custom polyclonal Abs against a protein immunogen.

Mice are observed daily, recording water and food consumption, abnormal clinical observations, mortality, and weekly weights. Blood is sampled and serum stored at baseline, then before boost and 2 weeks after boost, with terminal bleed via cardiac puncture. Serum is stored and spleen mononuclear cells are harvested and cryopreserved for the ELIspot and intracellular cytokine staining procedures.

Evaluating immunogenicity—analytic considerations and experiment planning. For each immunization strategy, groups of 6 animals are employed. The power analysis, dictating 6 animals/group, is based on a two-sided, two-sample t-test with hypothesized relative effect sizes, |μ1-μ2|/s, yielding at least 90% power to detect a relative effect size, |μ1-μ2|/s, of 2.7 between any two groups with a familywise type I error of 0.05, which accounts for multiple comparisons although, given the data set forth herein, the observed effects are expected to be far in excess of this threshold. Using groups of 6 animals therefore provides better than adequate power to identify the better immunization strategy in each pair.

For each of these comparisons, an evaluation to advance a particular immunization procedure to the next stage comparison is the production of IgG (Total) against the immunogen, but the ability of the new procedures to elicit production of specific IgG subtypes, IgM, and IgA, and CMI is also taken into account.

The experiments presented above are also repeated in an expanded form, with additional controls. The immune responses of mice immunized with wt E. coli (ME5000, the TMUG parental strain, as well as a commonly used E. coli strain, MG1655) [references 19, 20], transformed with the immunogen expressing plasmids and bacteria not transformed with plasmids as negative controls, are compared. GR E. coli TMUG strains, with genome reductions of 2.4%, 15.9%, and 29.7%, are compared employing the more extensive immune assays described above.

As an initial comparator for traditional immunization procedures, SC immunizations with the bacteria expressing the immunogens is employed, and SC immunizations with commercial recombinant HA and GFP. For the protein antigens the CFA prime, IFA for boosts [reference 47]) are employed. In addition, for these comparison experiments, to further support the ability of a standard immunization procedure to elicit a good humoral immune response, the HA and GFP test immunogens are conjugated to KLH using a widely available kit (e.g. AAT Bio ReadiLink KLH Conjugation Kit or Novus/Techne Imm-Link for carboxyl conjugation since the HA immunotag does not include S or M).

Evaluating immunogenicity—pairwise comparisons of immunization procedure enhancements. The data presented herein indicated, in a type of “dose-response” study, that immunization with bacteria expressing recombinant immunogen on the surface of the most highly GR E. coli strain (29.7% deleted) elicited much better immune responses than the wt or less deleted bacteria (see FIGS. 4A and 4B). Assuming the repeat experiments recapitulate the results, responses elicited by the largest deletion, 29.7%, GR E. coli expressing the immunogens and administered intranasally, pairwise, are compared with: a) SC immunization with the protein immunogen using the CFA prime/IFA boost regimen, b) SC immunization with wt E. coli expressing the immunogen, and c) SC immunization with the 29.7% GR E. coli expressing the immunogens. Given the data presented herein, and the benefits of intranasal immunization described above, it is hypothesized that the intranasal immunization with the GR E. coli vector yields the best immune responses.

The product of the experiments is a vaccination regimen that is used to show that the new procedures can rapidly yield the production of usable quantities of custom Abs.

Demonstrate Rapid Production of Polyclonal Antibodies in Rabbits Using Optimized Immunization Procedures. Since the ultimate goal of the project is to develop new procedures that enable the rapid production of custom Abs broadly useful for biomedical research, it is shown that the presently disclosed subject matter can rapidly produce custom Abs in the species most commonly used to produce new custom polyclonal Abs, the rabbit. Having optimized and refined the new immunization procedure in the mouse experiments as described herein above, it is then shown that the optimized procedure is effective in the rabbit. Chosen are two immunization procedures that yielded the highest mean Ab concentration elicited 14 days after the boost immunization and the procedures are used to immunize the rabbits, comparing the two best procedures with each other and with SC immunizations with killed whole bacteria vaccines, and with SC immunization using purified immunogen proteins.

In these experiments, a focus is made on the intranasal immunization of rabbits, in comparison with the typical CFA/IFA SC protein prime/boost schedules, given the results provided herein. If immunization via another route with the GR E. coli proves to be more effective, then that route is used. In any case, given the present results, shown in FIGS. 4A and 4B, with the evidence for greatly enhanced accessibility of the surface expressed immunogen on the GR E. coli, it is believed that expression of protein immunogens on the surfaces of GR E. coli elicit enhanced immune responses compared to non-GR bacteria or native protein.

New Zealand White rabbits are used, since this breed is the most commonly used breed to produce custom antibodies and is of medium-large size, enabling reasonable blood volume sampling.

Rabbit immunizations. Immunized are groups of 6 rabbits with the statistical considerations described above. For the rabbit studies, due to the larger size of the animals, 10⁹ cells are used for single immunizations. If the exp-inc immunizations in the mice yield significantly better responses, for the rabbits 10⁹, 3×10⁹, and 10¹⁰ cells per dose every other day are used.

Upon observation in the initial mouse studies that intranasal immunization yields the best immune responses, which is expected based on the presently disclosed data, this route will also be employed for the rabbit confirmatory studies. Intranasal immunization has been commonly used in rabbit studies, typically in a volume of 0.5 ml, administered by dripping by pipette into the nares of the rabbit held in an inverted position [references 48, 49].

For the comparison, rabbit SC immunizations are used, employing CFA/IFA prime-boost methods widely described in detail in standard reference works [reference 47], which procedures are uses in conducting the SC immunizations as reference for comparing the new GR E. coli methods, using the HA and GFP (conjugated, see above) test immunogens. For the comparison conventional protein immunizations, 200 μg for the prime and 100 μg for each boost are used. Boosts every 2 weeks for a total of 5 times are carried out, with both the conventional procedure and new GR E. coli-based immunizations.

Rabbit blood sampling. Blood samples are obtained via marginal ear vein venipuncture. Assuming a ˜5 Kg size for the typical New Zealand White, with a blood draw volume limit of 1% of body weight every 2 weeks, ˜5 ml are sampled at baseline, then after 2 weeks, before boosting, and 2 weeks after each boost at 2 week intervals. After the final boost sampling continues at 2 wk intervals for a total 6 months, to evaluate any additional maturation in the immune responses and to establish that the blood draws from the rabbits immunized according to the accelerated procedure can produce commercially and biomedically useful quantities of sera over a long time. At the end of the experiment, a terminal bleed is conducted following euthanasia to confirm that a useful maximum amount of sera from the rabbit immunized is produced in accordance with the presently disclosed subject matter.

Testing rabbit sera elicited by the new procedures. The main comparison between the standard procedure and the presently disclosed subject matter are Ab concentrations elicited against the HA and GFP immunogens, comparing the kinetics of Ab development in the conventional and new GR E. coli procedures. Humoral immunity is evaluated using the sandwich ELISA methods described above, using the appropriate HRP-conjugated anti-rabbit IgG (Total), IgG1, IgG2a, and IgA and IgM antibodies (Abcam, ThermoFisher, Sigma) in place of the anti-mouse used above. For the purposes of this EXAMPLE one evaluation is a comparison between the presently disclosed subject matter and conventional procedures at the 2-week and 4-week times after the first prime immunization, and the key evaluation is the comparison between the sera elicited from the rabbits using the new procedure at 2 and 4 weeks with the sera elicited from the rabbits using the conventional procedure at end of the experiment, 16 weeks after the prime immunization.

Testing rabbit cellular immune responses elicited by the new procedures. While a goal of the presently disclosed subject matter is the rapid production of biotechnologically effective antisera, also compared is the ability of the GR E. coli to stimulate cell-mediated immunity. Essentially the same techniques as described above for the study of mouse cell-mediated immunity are used, modifying the procedures to use the appropriate antibodies and reagents directed against the equivalent rabbit markers. For the rabbit experiments, given that much larger blood volumes can be obtained, peripheral lymphocytes are used, and the ELIspot assays are conducted on blood from each draw, not just the terminally obtained spleen cells.

Functional evaluation of sera elicited in rabbits. Since a goal of the presently disclosed subject matter is to develop a procedure to rapidly elicit highly effective custom polyclonal antibodies for use in a wide variety of biological research projects, it is confirmed that the sera elicited against the test immunogens in the rabbit are functional for techniques in which custom antisera are used. These include immunoblotting, flow cytometry, and immunofluorescence microscopy. For each of these applications the polyclonal Abs produced in accordance with the presently disclosed subject matter are compared with commercially available rabbit polyclonal Abs (e.g Abcam). For the immunoblotting evaluation, it is confirmed that the elicited rabbit sera can detect the HA and GFP antigens produced in the bacteria, run in parallel with commercially produced GFP and HA-immunotagged protein standards (e.g. Abcam, ThermoFisher), as in FIGS. 4A and 4B, using commercial HRP-conjugated anti-rabbit secondary Ab. For the flow cytometry experiments the ability of the elicited rabbit antisera to bind the bacteria as in FIGS. 4A and 4B is compared, but since many Abs in the polyclonal sera are likely directed against bacterial antigens, a relevant test is for the ability of the sera to stain mammalian cells expressing the test antigens. For the flow cytometry and IF experiments, commercially available stable cell lines expressing GFP and HA-fusion proteins (e.g. GenTarget, R&D/Biotechne, ATCC) are employed, using standard protocols recommended by the vendor of the commercial polyclonal rabbit Abs (e.g. https://www.abcam.com/protocols/indirect-flow-cytometry-protocol; https://www.abcam.com/protocols/immunocytochemistry-immunofluorescence-protocol) with commercially available anti-rabbit (e.g. ThermoFisher Invitrogen Goat anti-rabbit-Alexa 594). In the case of the flow cytometry studies using the GFP cell lines, the signal from GFP gating is directly compared with signal gating on the fluor labeling the anti-rabbit secondary Ab.

Potential alternatives. An aspect of the presently disclosed subject matter is that surface expression of immunogen on the GR E. coli is particularly immunogenic. It is believed that the presented (see FIGS. 4A and 4B) indicate that effective polyclonal Abs are rapidly elicited, and aspects of the presently disclosed subject matter are biologically plausible. An additional approach to enhancing immune response is also tested: exp-inc immunization schedule. There are additional methods to increase immunogenicity, which can be tested, if needed. Bacteria can be additionally administered with additional adjuvant, such as cholera toxin B subunit, AS03, AS04, and/or MF59, although care is needed in using lipophilic adjuvants that they do not excessively damage the bacterial outer membrane that holds the immunogen.

FIG. 6 illustrates the projected timeline. The first two years of the project are devoted to optimizing the immunization protocols in mice. The immunogen-expressing plasmids are constructed and tested, and a series of head-to-head comparisons of routes, immunogens, and schedules (single dose vs. exp-inc), comparing the induction primarily of humoral immune responses, are performed. Thereafter, how the best, optimized immunization methods developed in the mouse models work to rapidly produce custom polyclonal Abs in the rabbit, the principle source for custom polyclonal Abs for research purposes, are determined.

An immediate future downstream biotechnological application of the new technology is the accelerated production of mouse mAbs. Antigen production, purification, conjugation, immunization, and boosting for the production of mouse mAbs also occupy considerable time prior to fusion and hybridoma production. Shortening this time from months to weeks would significantly accelerate production of mouse mAbs to a similar extent that will occur with the production of custom polyclonal Abs.

A very rapid synthetic biology recombinant bacterial vaccine platform is useful for many clinical purposes, from the rapid development of prophylactic vaccines for infectious diseases to custom tumor antigen-directed cancer immunotherapy. From a basic biological research perspective, understanding why the GR E. coli are such highly effective immunogens yields insights helpful in many areas. For example, is the enhanced immunogenicity simply the result of increased immunogen accessibility, or do some of the gene products removed from the surfaces of the GR E. coli blunt the host response against the immunogens expressed on the bacterial surface, and if some of the gene products do blunt the host immune response, what are the responsible mechanisms? Understanding such potential mechanisms provides insights into the processes governing the assembly and maintenance of a wide range of host microbial communities.

### Example 5

Strategies for Producing Genome Reduced (Gr) Bacteria for Use in Immunotherapy

While impressive advances have been made in treating some cancers, other cancers still have dismal survival statistics. Immunotherapy offers great promise, but effective immunotherapy for many cancers is lacking. Further, current immunotherapeutic approaches are extremely expensive and hence inappropriate for global use, and there is an increasing recognition that much of the global burden of cancer falls on people living in the developing world.

The presently disclosed subject matter provides that expressing an antigen on the surfaces of Genome Reduced (GR) E. coli (bacteria that have a large (−30%) portion of their genomes deleted), using a Gram-negative autotransporter, and then making a Killed Whole Cell (KWC) bacterial vaccine from those bacteria, an unexpectedly potent immune response against the surface expressed vaccine antigen is elicited by the vaccine.

This EXAMPLE employs the presently disclosed subject matter to make a therapeutic cancer vaccine targeting the well-known tumor antigen survivin (see for example references 52, 100, and 101). KWC Gram-bacterial vaccines for infectious disease prophylaxis for diseases such as cholera are currently prequalified and stockpiled by WHO at a production cost of <$2/dose, indicating that a globally appropriate KWC GR E. coli therapeutic cancer vaccine could be commercially manufactured.

Wild type and GR E. coli that express survivin on the bacterial surface are prepared. KWC vaccines are prepared from the survivin-expressing GR E. coli. It is demonstrated that the vaccines are safe in mice and that cellular and humoral anti-survivin immune responses are elicited. It is further demonstrated that the vaccines have an anti-tumor effect in a mouse model. The antitumor response elicited by the GR E. coli is envisioned to be greater than that elicited by wild type (wt) E. coli expressing survivin.

Approach. A codon-optimized version of the survivin coding sequence is synthesized and cloned into a synthetic surface expression plasmid in accordance with the presently disclosed subject matter, and transformed into wt and GR (2.4%, 15.9%, 29.7%) E. coli, and expression is verified with immunoblots and flow cytometry. KWC vaccines are produced from the bacteria expressing survivin on their surfaces, and are used to immunize mice. The immunizations are evaluated by assessing anti-survivin humoral immune responses by ELISA and cellular immune responses by ELIspot and flow cytometrically for antigen-specific T-cell frequency, proliferative capacity, cell surface immunophenotyping, and intracellular cytokine production profiles. If needed, immunization schedules and immunogen preparations are adjusted to enhance immune responses, for example using exponential-increasing immunization schedules for the GR E. coli. The ability of the vaccines to yield anti-tumor effects in a mouse tumor treatment model system is also tested.

This EXAMPLE provides a proof-of-concept for a low cost, globally appropriate KWC GR E. coli therapeutic cancer vaccine targeting survivin, which is in itself be a significant advance. However, since the presently disclosed subject matter provides a synthetic biology solution, the platform can be easily modified to produce analogous vaccines targeting many other tumor antigens. The presently disclosed subject matter can also be coupled with a molecular analysis of the tumor, for example using RNAseq, to rapidly produce custom combination therapeutic cancer vaccines tailored to each cancer's tumor antigen expression profile, offering a new, inexpensive, globally appropriate immunotherapy option for many cancers.

Cancer immunotherapy. Immunotherapy holds great promise for cancer therapeutics, but inexpensive cancer immunotherapy, easily modifiable to target multiple tumor-associated antigens (TAAs) and customizable for a particular patient's cancer, is lacking [reference 50]. This is a particular problem for global applications [reference 51]. Immunotherapy that has been effective to date, like checkpoint inhibition, administration of tumor TAA-directed antibodies, or adoptive T cell therapies, are costly and/or difficult to customize.

Survivin as an immunotherapy target. The TAA survivin, is an immunotherapeutic target overexpressed in many tumors, including CNS tumors, sarcomas, Wilms tumor, neuroblastoma, AML, and B-cell lymphomas, notably Burkitt lymphoma, a major problem in the developing world [references 52, 53]. Survivin is an attractive immunotherapy target, but survivin-directed immunotherapies have not necessarily met their initial promise. New approaches to immunotherapeutically target survivin, and other TAAs, need to be developed [references 54, 55]. Survivin makes an excellent proof-of-principle target to develop new cancer immunotherapeutics.

Recombinant bacterial immunogens. Fixed, whole cell bacterial vaccines have long been recognized as effective ways to elicit desired immune responses. Killed whole cell bacterial vaccines have several advantages: they are inexpensive and easy to produce, and are auto-adjuvanting, carrying a large number of PAMPs. Formalin fixed Killed Whole Cell (KWC) vaccines, have been WHO-qualified as prophylactic vaccines for S. Typhi and cholera [references 56, 57].

Gram-negative autotransporters. A bacterial surface expression system offers a helpful way to place many immunogens on the surface of bacteria. Gram-Autotransporter (AT) (also termed Autodisplay or Type 5 Secretion System) proteins are a protein family that can place recombinant proteins into Gram-bacterial outer membranes, with one region anchored in the membrane lipid bilayer and another exposed to extracellularly [references 58-61]. AT proteins have 3 key domains: An N-terminal signal sequence that directs protein across the inner membrane via a secA mechanism, a C-terminal β-barrel that inserts into the Gram-OM, yielding a pore-like structure, and a central passenger protein domain that transits through the β-barrel pore to be exposed extracellularly, attached to the β-barrel, which remains anchored in the OM. Native passenger protein coding sequence can be replaced with sequence encoding another protein, yielding a recombinant AT protein. The AT thus ‘displays’ recombinant passenger protein to the environment, anchored in the OM lipid bilayer. About 2×10⁵ recombinant proteins can be placed on each cell's surface [reference 61]. Bacterial surface expression of antigens (Ags) offers the additional theoretical advantages of extreme multivalency and increased avidity. AT-expressed Ags have the additional advantage that translation and export to the cell surface of very tightly coupled, abrogating any concerns for formation of insoluble intracellular aggregates, and the AT expression process has inherent chaperonin-like activity. AT-derived vaccine Ags have elicited humoral and cellular immunity, and showed protective effects against a pathogen [references 62, 63]. However, despite considerable promise and much published work, AT-based vaccines have not yet been used clinically in either humans or animals, perhaps because they were not sufficiently immunogenic [reference 64]. Developing technology to enhance the immunogenicity of vaccine Ags expressed on the surfaces of Gram-negative bacteria would enable the substantial promise of vaccine immunogen expression by Gram-negative ATs to be fulfilled.

Genome reduced E. coli. Several groups have asked this fundamental question: What is the minimal complement of genes that a bacterium such as E. coli requires to live and replicate? The Tokyo Metropolitan University Group (TMUG) [references 65, 66] has shown that they can delete 29.7% of the E. coli genome and still have a viable, albeit slow growing organism.

This EXAMPLE discloses that placing recombinant Ags on the surface of genome reduced (GR) E. coli using a Gram-AT expression cassette elicits a greatly enhanced immune response against that Ag compared to the response elicited by that same Ag expressed on the surface of wt E. coli [references 65, 66], which is ideal for the induction of immune responses against tumor Ags.

Data. As shown herein above, to count the proteins eliminated from the surface of GR E. coli the genes in each TMUG GR deletion (LD5510, LD5119, and LD5125) using the National BioResource Project E. coli Strain website (https://shigen.nig.acjp/ecoli/strain/resource/longDeletion/lddTableInfo), with additional information from EcoCyc (https://ecocyc.org/), and UniProt (https://www.uniprot.org/): gene name, protein name, location, function, gene ontology, and other notes about the gene were listed. It was found that almost 200 gene products were eliminated from the bacterial surface in the 29.7% GR strain. The analysis suggested that removing proteins from the surface of the bacteria would indeed make the Ag more accessible to the immune system, or make more recombinant immunogen to be placed on the bacterial surface. In addition, commensal enteric organisms, like E. coli, must live within the GI tract without provoking a host immune response that could lead to their elimination. Deleting proteins from the bacterial surface might then also disrupt mechanisms that the enteric bacteria evolved to blunt host immune responses, providing an additional rationale for the effectiveness of an immunogen made from GR E. coli expressing immunogen on their surfaces.

Gram-AT recombinant expression systems for rapid Ab production immunizations. As discussed herein above, a plasmid, pRIAIDA (FIG. 1; SEQ ID NO: 1) that has a rhamnose-inducible AIDA-I Gram-AT expression cassette was produced, with a cloning site, flanked by a trypsin site to evaluate surface expression. In initial experiments, the widely used influenza virus HA tag (YPYDVPDYA; SEQ ID NO: 2) was placed in the surface expression cassette to make pRIAIDA-HA. FIG. 1 shows the map of the plasmid (FIG. 1). A trypsination experiment confirming that an HA immunotag resides on the exterior of the bacteria when expressed in an AIDA-I expression cassette that includes a trypsin site between the expressed tag and the β-barrel (FIG. 1).

Evaluation of antibody binding to the GR E. coli expressing a test immunogen on their surfaces and the ability of the GR E. coli expressing a test immunogen on their surfaces to elicit immune responses. As discussed hereinabove, pRIAIDA-HA was transformed into wt parental E. coli and three GR E. coli from the TMUG collection. Binding of a commercial anti-HA mAb to the bacteria was evaluated via flow cytometry (FIG. 2). It was found that the ability of the bacteria to bind the anti-HA mAb increased significantly as the fraction of the genome deleted increased. Then, the ability of the GR E. coli to elicit an immune response was tested. Mice were immunized intranasally with 10⁸ formalin fixed bacteria. IN immunization directly exposes M and dendritic cells to the immunogen. There are abundant nasopharyngeal lymphoid tissues with large numbers of other Ag presenting cells, like macrophages, and many T cells and B cells. IN immunization can induce potent tissue-resident effector and effector memory CD8+ T cell immunity [reference 73]. Since once of the goals of the project, and the FOA, is to develop cancer therapeutics that are highly appropriate for lower resource settings, administering immunotherapies IN would be helpful, since no needles would be required and the tumor vaccine could be administered by personnel with less training.

After 2 weeks blood was collected from the immunized mice and tested the sera using a purpose-built sandwich ELISA, with commercial anti-HA mAb as a standard. It was found that IN immunization with GR E. coli expressing the test immunogen on their surfaces could elicit the production of large amounts of Abs in only 2 weeks, and that the ability of the bacteria expressing the test Ag to elicit production of the Ab increased substantially in bacteria with more genes deleted (FIG. 3). Immunoblots were prepared that show that about the same amount of HA is produced per cell regardless of extent of the genome reduction. While it is not desired to be bound by any particular theory of operation, the data support that the increased binding and immunogenicity is due to either increased Ag accessibility in the GR strains, or an absence of immunoinhibitory surface structures or both.

Developing assays for anti-survivin immune responses. Using commercial anti-survivin mAbs with HRP-conjugated goat anti-mouse secondary antibody, assayed using the Tropix CSPD luminescence system, anti-survivin Abs were shown to be detectable to the 1 ng/ml level, below the physiologic level of antibodies elicited by approved vaccine Ags. Plasmids were produced expressing rhamnose-inducible his₆-tagged survivin cytoplasmically, isolating endotoxin-depleted survivin proteins from bacterial extracts. To show anti-survivin immune responses could be detected, mice were immunized IM with 50 ug survivin with montanide and CpG adjuvants. Blood was sampled before and after immunization, using the ELISA, and tested spleen mononuclear cells at the end of the experiment and detected responses.

Scientific Premise. The scientific premise underlying the project holds a killed whole cell vaccine made from GR E. coli expressing a TAA, like survivin, on their surfaces via an AT expressing cassette elicit a potent immune response against the tumor that have useful anti-tumor effects. The premise is supported by data described above.

Overview. A survivin-expressing plasmid is constructed and transformed into wt and GR E. coli, KWC vaccines are made from the bacteria, and the ability of the vaccines to elicit humoral and cellular immune responses is tested. If the initial responses are insufficient, additional strategies are used to enhance immune responses. The KWC GR survivin vaccines are further tested for effect in a mouse tumor model.

Design of expression cassettes, surface expression and bacterial toxicity testing. An E. coli codon-optimized version of an appropriate survivin coding sequence is synthesized and cloned it into the expression plasmid, pRIAIDA (FIG. 1). The derivative, pRIAIDA-S, is transformed into wt non-deleted and GR (2.4%, 15.9%, 29.7%) E. coli [references 65, 66], verifying expression by flow cytometry and immunoblot with/without trypsin treatment. Optimum induction of expression is determined, following growth by OD₆₀₀, to determine maximum expression possible without compromising replication. The number of survivin molecules on the surface of each bacteria is determined using immunoblots and survivin protein standards. A target is at least about 2×10⁵ molecules/bacteria, and which has been achieved with other Ags.

Production of immunogens—whole GR E. coli. The methods are those that yielded the data (FIG. 3). Bacteria are grown in LB broth with optimized rhamnose induction, monitoring growth by OD₆₀₀. Bacteria are collected by centrifugation, washed and inactivated with formalin. Aliquots are stored at −80° C. in PBS/10% glycerol, confirming survivin expression by flow cytometry on thawed stocks. Surface expression is also confirmed.

Immunizations. Immediately before immunization, the KWC preparations are thawed on ice and washed in PBS. 6 week old mice (CB6F1/J, Jackson) are immunized IN with 10⁸ cells in 50 ul PBS, the protocol used to obtain the data of FIG. 3, boosting at 2 and 4 weeks. We established 10⁸ cells as a tolerated, immunogenic dose with HA-expressing bacteria (FIG. 3). This data showed that highly GR E. coli elicited an unexpectedly potent immune response against HA expressed on the bacterial surface. IM immunizations (FIGS. 4A and 4B) with recombinant survivin are used as a positive control comparator. Mice immunized with E. coli expressing the HA immunogen are used as a negative control comparator. Mice are observed daily, recording water and food consumption, abnormal clinical observations, and mortality, with weekly weights. Blood is sampled and serum is stored at baseline, then before boosts and every 2 weeks for 8 weeks, with terminal bleed via cardiac puncture. Serum is stored and spleen mononuclear cells are harvested and cryopreserved.

Evaluating immunogenicity—humoral immunity. To evaluate Abs elicited by the KWC vaccine in the experiments, a sandwich ELISA is constructed by binding commercial anti-HA (or anti-survivin) mAb (Invitrogen) to blocked, streptavidin-coated strips (Pierce), followed by incubation with commercially produced HA peptide or immunotag-purified recombinant bacterially-produced survivin, followed by commercial anti-HA mAb (Invitrogen) with HRP-conjugated goat anti-mouse secondary antibody, assayed using the Tropix CSPD luminescence system. The same methods are used to assay for anti-survivin Abs elicited by the KWC GR E. coli survivin vaccines. For the experiments evaluating immune responses in this EXAMPLE, IgG subclasses (IgG1, IgG2a, IgG (Total)), and IgA and IgM with the appropriate HRP-conjugated anti-mouse class and subclass mAbs are quantified.

Evaluating immunogenicity—cell-mediated immunity. Cell-mediated immunity are evaluated by ELIspot assays, performing assays according to kit manufacturer's instructions (Mabtech). Spleen cells are plated, stimulating with overlapping 15-mer survivin peptide mix (or HA, incubating for 24 h. Cells are removed, plates washed, and incubated with biotinylated detection antibody, then incubate with streptavidin-ALP, followed by substrate solution (BCIP/NBT-plus). Spots are counted in control wells, subtracting this from the spots counted in the test wells.

In addition to ELIspot assays, the assessment of Ag specific T-cell frequency, proliferative capacity, cell surface immunophenotyping, and intracellular cytokine production profiles by flow cytometry is combined to characterize T cell responses. Cells [reference 74] challenged with immunogens are evaluated with a polychromatic (12 color) flow cytometric panel to determine frequencies of survivin (or positive control HA) positive cells, and T_{EM} or T_{CM} phenotypes are characterized, since T_{CM} cells vs. T_{EM} cells [references 75-77], which might imply the induction of longer-term immunity, might be helpful for additional boosting and distinguishing between different immunization strategies. For defining T_{EM} and T_{CM} cells, markers are used as recommended by the Human Immunology Project [reference 77]: CD3, CD4, CD8, CCR7, CD45RA. To assess functionality of the response, intracellular cytokine production and proliferative capacity are measured, performing intracellular cytokine staining for IL2, IFN-γ, and TNF-α. Proliferation by intracellular staining for Ki-67 is assessed, which is expressed in cells in S, G₂, and M phases, but not G₀ or G₁ [reference 79]. A fixable amine reactive viability dye is used to eliminate evaluation of dead cells. Non-T cells are identified with a cocktail including anti-CD19, CD14, CD16, CD56, CD11c and CD11b Abs. At least 100,000 events on a BD 4 laser 17 color FORTESSA™ flow cytometer are acquired. Data using FlowJo10 (Treestar) software are analyzed with this gating strategy: 1) Gate for single cells using a FSC area vs. FSC height plot, 2) Gate for live T cells using a CD3 vs. Viability/dump channel (lineage cocktail) plot, 3) Gate for Ag specific CD8+ T cells using CD8 vs. Pentamer plot, 4) Phenotype T_{EM} and T_{CM} using CCR7 vs. CD45RA plot, and 5) Evaluate intracellular cytokine profiles and Ki-67 positivity within the T_{EM} and T_{CM} cell populations.

Evaluating immunogenicity—analytic considerations and experiment planning. For each immunization strategy groups of 6 animals are used. The power analysis, dictating 6 animals/group, is based on a two-sided, two-sample t-test with hypothesized relative effect sizes, |μ1-μ2|/s, yielding at least 90% power to detect a relative effect size, |μ1-μ2|/s, of 2.7 between any two groups with a familywise type I error of 0.05, which accounts for multiple comparisons although, given the data disclosed herein, the observed effects are expected to be far in excess of this threshold. Using groups of 6 animals therefore provides better than adequate power to identify the better immunization strategy in each pairwise comparison.

Enhancing immunogenicity—alternative approaches. This Example is conservatively powered to detect 2.7-fold differences, the immunogenicity target for the new KWC recombinant GR E. coli survivin vaccine is a 10-fold increase in in Ab concentration measured by ELISA and a 10-fold increase in ELIspot counts comparing the GR bacteria with the wt parental bacteria and comparing the GR E. coli vaccine to conventional IM immunization, an essentially arbitrary choice. But, the presently disclosed subject provides a cancer vaccine that is substantially more than incrementally more immunogenic than that achievable with current technology, and a log-fold increase represents a biologically meaningful goal.

If a log-fold increase in immune responses over conventional IM protein Ag immunization alone is not achieved with the KWC GR E. coli vaccine, at a minimum, an additional strategy to enhance immunogenicity is employed: exponential-increasing immunization schedules.

Exponential increasing (exp-inc) immunization schedules. Repeated immunizations with exp-inc amounts of immunogens can yield a dramatically improved immune response, with a >10-fold increase in Ab concentrations [reference 89]. If needed, an exp-inc schedule, with doses of 10⁸, 3×10⁸, and 10⁹ cells on alternating days is used. The hypothesized reason for the enhanced response is that the exp-inc Ag dosing schedule creates signals to the host immune system similar to those initiated by serious infections that threaten the host, with the enhanced response promoted by prolonged, greater amounts of Ag present in lymph nodes acting to improve antibody maturation. Exp-inc immunization has also been observed to induce more Tfh cells and germinal center B-cells.

Additional strategies. Additional approaches to increasing immunogenicity further include additional adjuvants (e.g. cholera toxin B subunit, CpG; AS03, AS04, and/or MF59 (although care is needed in using lipophilic adjuvants so that they do not excessively damage the bacterial outer membrane that holds the immunogen) and overexpression of nucleic acid PAMPs like ds RNA).

Demonstrate that the vaccines have an anti-tumor effect in a mouse model. MethA sarcoma cells [references 90-92] are tested in BALB/c mice and EL4 tumor cells in C57BL/6 mice [references 93-95], as initial, well-established mouse tumor models tumors representing therapeutically problematic cancers (sarcomas and lymphomas). 5×10⁵ tumor cells are injected subcutaneously, comparing tumor growth in mice immunized with survivin-expressing bacteria and controls, measuring the two greatest extent axes, expressed as mm². Vaccines are administered simultaneously with tumor cell injection, boosting at 2 and 4 weeks. Subsequently the studies are repeated, delaying the immunizations until 1 and 2 weeks after tumor cell injection. With input from the Biostatistics Core, 8 animals per treatment group are planned, which yields 85% statistical power to detect a standardized effect size of 3 between any two treatment groups per experiment using a two-sided test with a family-wise type I error of 0.05. The family-wise type 1 error of 0.05 accounts for multiple comparison testing in the final analysis. Animal health status is monitored and recorded in a log using a body condition score system (i.e., BCS 1-5). Animals are monitored twice daily, with daily weights. Animals with BCS<2 or >20% body weight loss are euthanized. Tumor size are measured thrice weekly. The primary endpoint comparisons tumor size at each timepoint and secondarily time to death/euthanasia (Kaplan-Meier, significance via log-rank test). Animals are evaluated histologically after euthanasia, and at the first time point where a statistically significant difference in tumor size is observed.

Vaccine immunology studies. Mouse immune responses to vaccine in the tumor studies are evaluated as above for blood and splenocytes, obtained at the end of the experiment. In addition, infiltration of CD3+ cells into tumor tissues and of cytotoxicity/necrosis is evaluated, essentially as described [reference 96].

The vaccine is tested in additional animal tumor models, followed by considerations of advancing to early phase 1 clinical trials. Additional work also includes pre-clinical toxicity and immunogenicity studies in other species, and development of materials needed to advance to a clinical trial, e.g. GMP production and QA plans, assay SOPs to evaluate vaccine immune responses. An IN KWC vaccine minimizes many safety and regulatory questions [references 97, 98], with no concerns about replication of potential pathogens, reversion of attenuated organisms to pathogenic phenotypes, or anti-vector immune responses rendering boosting ineffective, unlike live viral or bacterial vectors. A KWC E. coli vaccine is comparable to approved KWC Gram-negative bacterial vaccines (e.g. for typhoid fever, cholera).

The presently disclosed subject matter is also used to create therapeutic cancer vaccines for other TAAs [reference 55]. New GR E. coli vaccines are produced, ready for initial testing in animals, for essentially any tumor TAAs in 10-14 d (5-7 d for gene synthesis, 2 d for cloning, 1 d for transformation, 1-2 d for bacterial inactivation and vaccine preparation). A similar timeline would hold for custom therapeutic cancer vaccines. Of course, induction of immune responses by the therapeutic vaccines can be further amplified by employing checkpoint inhibitors [reference 99]. Overall, the presently disclosed subject matter offers significant promise for the rapid production of highly immunogenic, inexpensive, therapeutic cancer vaccines particularly appropriate for global use.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicant reserves the right to challenge the accuracy and pertinence of any cited reference.
Altschul et al. (1990a) J Mol Biol 215:403-410.
Altschul et al. (1990b) Proc Natl Acad Sci USA 87:14:5509-13.
Altschul et al. (1997) Nucleic Acids Res 25:3389-3402.
Bodanszky & Bodanszky (1984) in The Practice of Peptide Synthesis, Springer-Verlag, New York, N.Y.
Chou & Fasman (1974) Biochemistry 13:222-245.
Chou & Fasman (1978) Ann Rev Biochem 47:251-276.
Chou & Fasman (1979) Biophys J 26:367-384.
Colligan et al. (eds.) (1994-2004) Current Protocols in Immunology, John Wiley & Sons, Inc., New York, N.Y.
Datsenko & Wanner (2000) Proc Natl Acad Sci USA 97:6640-6645.
Deutscher et al. (ed.) (1990) Guide to Protein Purification, Harcourt Brace Jovanovich, San Diego, Calif.
Devereux et al. (1984) Nucl Acids Res 12:387.
Gerhardt et al. (eds.) (1994) Methods for General and Molecular Bacteriology, American Society for Microbiology, Washington, D.C., p. 574.
Gross & Mienhofer (eds.) (1981) The Peptides, Vol. 3, Academic Press, New York, N.Y., pp. 3-88.
Hashimoto et al. (2005) Mol Microbiol 55:137-149.
Karlin & Altschul (1990) Proc Natl Acad Sci USA 87:2264-2268.
Karlin & Altschul (1993) Proc Natl Acad Sci USA 90:5873-5877.
Kittlesen et al. (1998) J Immunol 60:2099-2106.
Kyte & Doolittle (1982) J Mol Biol 157:105-132.
Morgan & Gainor (1989) Ann Rep Med Chem 24:243-252.
PCT International Patent Application Publication Nos. WO 1999/54464; WO 2004/110486; WO 2006/012539.
PCT International Patent Application Serial No. PCT/US2005/026102.
Rapp & Kaufmann (2004) Int Immunol 16:597-605.
Remington's Pharmaceutical Sciences, 18th ed. (1990) Mack Publishing, Easton, Pa.
Spatola (1983) Chemistry and Biochemistry of Amino Acids, Peptides and Proteins, Vol. VII. Weinstein (ed.). Marcel Dekker, Inc., New York, N.Y., pp. 267-357.
Stewart et al. (1984) in Solid Phase Peptide Synthesis, 2nd Edition, Pierce Chemical Company, Rockford, Ill.
U.S. Patent Application Publication No. 2008/0193469.
U.S. patent application Ser. No. 11/572,453.
Welters et al. (2004) Vaccine 23:305-311.
Zugel (2001) Infect Immun 69:4164-4167.

### REFERENCES CITED IN EXAMPLES 4 AND 5

1. Onodera et al. (2008) J Immunol 180(2):907-13.
2. Hasbold et al. (1998) Eur J Immunol 28(3):1040-51.
3. Stavnezer et al. (2008) Annu Rev Immunol 26:261-92. PMC2707252
4. Roost et al. (1995) Proc Natl Acad Sci USA 92(5):1257-61. PMC42498
5. Newman et al. (1992) J Immunol 149(10):3260-72.
6. Hangartner et al. (2006) Nat Rev Immuno. 6(3):231-43.
7. Hidalgo et al. (2003) Pediatr Infect Dis J 22(6):570-2.
8. Thomsen et al. (1997) Int Immunol 9(11):1757-66.
9. Kilpatrick et al. (1997) Hybridoma 16(4):381-9.
10. Berry et al. (2003) Hybrid Hybridomics 22(1):23-31.
11. Shata et al. (2000) Mol Med Today 6(2):66-71.
12. Ulmer et al. (2006) Nat Biotechnol 24(11):1377-83.
13. van Bloois et al. (2011) Trends Biotechnol 29(2):79-86. PMID: 21146237
14. Nicolay et al. (2015) Crit Rev Microbiol 41(1):109-23.
15. Henderson et al. (2000) Trends Microbiol 8(12):529-32. PMID: 11115743
16. Jose & Meyer (2007) Microbiol Mol Biol Rev 71(4):600-19. PMC2168652
17. Yang et al. (2010) Vaccine 28(15):2735-42. PMID: 20105428
18. Pompa-Mera et al. (2011) Exp Parasitol 129(4):393-401. PMID: 21907709
19. Kato & Hashimoto (2007) Mol Syst Biol 3:132. PMC1964801
20. Hashimoto et al. (2005) Mol Microbiol 55(1):137-49. PMID: 15612923
21. Zaman et al. (2013) Drug Deliv Transl Res 3(1):100-9. PMC3539070
22. Yusuf & Kett (2017) Hum Vaccin Immunother 13(1):34-45. PMC5287317
23. Nizard et al. (2017) Nat Commun 8:15221. PMC5458068
24. Riese et al. (2014) Expert Opin Drug Deliv 11(10):1619-34.
25. Jabbal-Gill (2010) J Drug Target 18(10):771-86.
26. Davis (2001) Adv Drug Deliv Rev 51(1-3):21-42.
27. Morabito et al. (2017) Mucosal Immunol 10(2):545-54. PMC5123975
28. Russell (2002) Am J Reprod Immunol 47(5):265-8.
29. Chen et al. (2010) Proc Natl Acad Sci USA 107(7):3099-104. PMC2840271
30. Frazer & Curtiss (1975) Curr Top Microbiol Immunol 69:1-84.
31. Farley et al. (2016) J Bacteriol 198(8):1186-95. PMC4859596
32. Carleton et al. (2013) Nat Commun 4:1590. PMC3693737
33. Jain et al. (2006) J Bacteriol 188(13):4841-50. PMC1483012
34. Tam et al. (2016) Proc Natl Acad Sci USA 113(43):E6639-E48. PMC5086995
35. Li et al. (2008) Biotechnol Bioeng 99(2):485-90.
36. de Boer et al. (1989) Cell 56(4):641-9.
37. Datsenko & Wanner (2000) Proc Natl Acad Sci USA 97(12):6640-5. PMC18686
38. LaBreck et al. (2019) J Bacteriol. January 28; 201(4)-18.
39. Sowa et al. (2009) Cell 138(2):389-403. PMC2716422
40. Huang et al. (2012) Nature 491(7424):406-12. PMC4854285
41. Maecker et al. (2005) BMC Immunol 6:17. PMC1190174
42. Klebanoff et al. (2006) Immunol Rev 211:214-24. PMC1501075
43. Klebanoff et al. (2005) Proc Natl Acad Sci USA 102(27):9571-6.1172264
44. Seder & Ahmed (2003) Nat Immunol 4(9):835-42.
45. Maecker et al. (2012) Nat Rev Immunol 12(3):191-200. PMC3409649
46. Gerdes et al. (1984) J Immunol 133(4):1710-5.
47. Greenfield (ed.) (2013) Antibodies: A Laboratory Manual. 2nd Ed. Cold Spring Harbor Laboratory Press, Cold Spring Harbor, N.Y.
48. Shoemaker et al. (2005) Infect Immun 73(8):5031-8. PMC1201206
49. Oliveira et al. (2007) Expert Rev Vaccines 6(3):419-29.
50. Majzner et al. (2017) Cancer Cell. 2017; 31(4):476-85.
51. Roe (2017) JAMA Oncol. 2017; 3(9):1169-70.
52. Garg et al. (2016) Cancer Cell Int. 2016; 16:49. PMC4917988
53. Fangusaro et al. (2006) Pediatr Blood Cancer. 2006; 47(1):4-13.
54. Altieri (2013) Cancer Lett. 332(2):225-8. PMC3695618
55. Melief et al. (2015) J Clin Invest. 125(9):3401-12. PMC4588240
56. Shata et al. (2000) Mol Med Today. 6(2):66-71.
57. Ulmer et al. (2006) Nat Biotechnol. 24(11):1377-83.
58. van Bloois et al. (2011) Trends Biotechnol. 29(2):79-86. PMID: 21146237
59. Nicolay et al. (2015) Crit Rev Microbiol 41(1):109-23.
60. Henderson et al. Trends Microbiol. 8(12):529-32. PMID: 11115743
61. Jose & Meyer (2007) Microbiol Mol Biol Rev. 71(4):600-19. PMC2168652
62. Yang et al. (2010) Vaccine. 28(15):2735-42. PMID: 20105428
63. Pompa-Mera et al. (20110 Exp Parasitol. 129(4):393-401. PMID: 21907709
64. Nicolay et al. (2015) Crit Rev Microbiol 41(1):109-23.
65. Kato & Hashimoto (2007) Mol Syst Biol. 3:132. PMC1964801
66. Hashimoto et al. (2005) Mol Microbiol. 55(1):137-49. PMID: 15612923
67. Zaman et al. (2013) Drug Deliv Transl Res. 2013; 3(1):100-9. PMC3539070
68. Yusuf & Kett (2017) Hum Vaccin Immunother. 13(1):34-45. PMC5287317
69. Nizard et al. (2017) Nat Commun. 8:15221. PMC5458068
70. Riese et al. (2014) Expert Opin Drug Deliv. 11(10):1619-34.
71. Jabbal-Gill (2010) J Drug Target. 18(10):771-86.
72. Davis (2001) Adv Drug Deliv Rev. 51(1-3):21-42.
73. Morabito et al. (2017) Mucosal Immunol. 10(2):545-54. PMC5123975
74. Maecker et al. (2005) BMC Immunol. 6:17. PMC1190174
75. Klebanoff et al. (2006) Immunol Rev. 211:214-24. PMC1501075
76. Klebanoff et al. (2005) Proc Natl Acad Sci USA. 102(27):9571-6.1172264
77. Seder & Ahmed (2003) Nat Immunol. 4(9):835-42.
78. Maecker et al. (2012) Nat Rev Immunol. 12(3):191-200. PMC3409649
79. Gerdes et al. (1984) J Immunol. 133(4):1710-5.
80. Chen et al. (2010) Proc Natl Acad Sci USA. 107(7):3099-104. PMC2840271
81. Frazer & Curtiss (1975) Curr Top Microbiol Immunol. 69:1-84.
82. Farley et al. (2016) J Bacteriol. 198(8):1186-95. PMC4859596
83. Carleton et al. (2013) Nat Commun. 4:1590. PMC3693737
84. Jain et al. (2006) J Bacteriol. 188(13):4841-50. PMC1483012
85. de Boer et al. (1989) Cell. 56(4):641-9.
86. Datsenko & Wanner (2000) Proc Natl Acad Sci USA. 97(12):6640-5. PMC18686
87. LaBreck et al. (2019) J Bacteriol. 2019 Jan. 28; 201(4). pii: e00374-18. doi: 10.1128/JB.00374-18.
88. Sowa et al. (2009) Cell. 138(2):389-403. PMC2716422
89. Tam et al. (2016) Proc Natl Acad Sci USA. 113(43):E6639-E48. PMC5086995
90. Finkelstein et al. (2012) Immunotherapy. 4(3):283-90. PMC4501768
91. Nagaraj et al. (2007) J Immunother. 30(2):169-79.
92. Sturmhoefel et al. (1999) Cancer Res. 59(19):4964-72.
93. Vincent et al. (2010) Cancer Res. 70(8):3052-61.
94. Qin et al. (2014) Nat Med. 20(6):676-81. PMC4048321
95. Kavunja et al. (2017) Cancer Immunol Immunother. 66(4):451-60. PMC5470645
96. Zhu et al. (2007) Vaccine. 25(46):7955-61.
97. Delany et al. (2014) EMBO Mol Med. 6(6):708-720.
98. Unnikrishnan et al. (2012) Curr Opin Immunol. 24(3):337-42.
99. Dhanak et al. (2017) Cell Chem Biol. 24(9):1148-60.
100. Chen et al. (2016) J Cancer. January 10; 7(3):314-23.
101. Warner et al. (2020) Stem Cell Rev Rep. October; 16(5):828-852

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A modified bacterium or derivative thereof having a reduced number of expressed genes and comprising an antigen, optionally an antigen on a surface of a membrane or derivative thereof, wherein the bacterium induces an enhanced immune response against the antigen when administered to a subject as compared to an immune response that would have been induced in the subject by a bacterium of the same strain that has a full complement of expressed genes.

2. The modified bacterium of claim 1, wherein reducing and/or eliminating expression of one or more gene in the bacterium yields the enhanced immunogenicity.

3. The modified bacterium of claim 1 or claim 2, wherein the bacterium is a Gram-negative bacterium, optionally a member of the Enterobacteriaceae.

4. The modified bacterium of any one of claims 1-3, wherein the bacterium is an E. coli.

5. The modified bacterium of any one of claims 1-4, wherein the reduced number of expressed genes comprises a reduction of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes.

6. The modified bacterium of claim 5, wherein the reduced number of expressed genes comprises a reduction of expressed genes selected from the group consisting of at least about 2.4%, at least about 15.9%, and at least about 29.7%.

7. The modified bacterium of any one of claims 1-6, wherein the antigen is put on the surface of the bacterium by an approach selected from the group consisting of expression by the cell itself, covalent or non-covalent association with the outer membrane, and combinations thereof.

8. The modified bacterium of claim 7, comprising an autotransporter (AT) expression vector encoding the antigen, wherein the expression on the surface is provided by the AT expression vector.

9. The modified bacterium of claim 8, wherein the autotransporter expression vector comprises a codon optimized sequence encoding the antigen.

10. The modified bacterium of claim 8 or claim 9, wherein the AT expression vector comprises a monomeric autotransporter vector or a trimeric autotransporter vector.

11. The modified bacterium of any one of claims 1-10, wherein the antigen is derived from a microbe.

12. The modified bacterium of any one of claims 1-11, wherein the antigen is derived from a cancer, or a target of an inappropriate or undesirable immune response, or a component of the host immune system, such as (but not exclusively), a host immune system component that, when targeted for destruction or inactivation or activation, alter an undesirable immune response.

13. A method for producing an antibody in a subject, the method comprising providing a modified bacterium according to any one of claims 1-12 and administering the modified bacterium to a subject in an amount and via a route sufficient to produce an antibody in the subject against the antigen expressed by the modified bacterium, optionally wherein the production of the antibody is enhanced in the subject as compared to an immune response produced in a subject by a bacterium of the same strain that has a full complement of expressed genes and that expresses the antigen on its surface.

14. The method of claim 13, comprising administering the modified bacterium to the subject intranasally, transmucosally, including but not limited to orally, rectally, and vaginally; subcutaneously, intradermally, intramuscularly, other parenteral routes, or any combination thereof.

15. A vaccine composition comprising a modified bacterium according to any one of claims 1-12 and a pharmaceutically acceptable carrier, optionally wherein the vaccine compositions further comprises one or more adjuvants.

16. The vaccine composition of claim 15, wherein the modified bacterium is a live attenuated bacterium or a killed whole cell bacterium, or derivatives or fragments thereof.

17. The vaccine composition of any one of claims 15 and 16, wherein the vaccine composition is adapted to be administered orally, rectally, vaginally, intra-nasally, parenterally, intradermally, subcutaneously, or intramuscularly.

18. The vaccine composition of any one of claims 15-17, wherein the vaccine composition further comprises an adjuvant.

19. A method for vaccinating a subject in need thereof, the method comprising providing a vaccine composition according to any one of claims 15-18 and administering the vaccine composition to the subject.

20. A method for treating a cancer or inappropriate immune responses or expression or production of a deleterious material in a subject in need thereof, the method comprising providing a vaccine composition according to any one of claims 13-15 and administering the vaccine to the subject.

21. The method of claim 19 or claim 20, wherein the vaccine composition is administered orally, rectally, vaginally, intra-nasally, parenterally, intradermally, subcutaneously, or intramuscularly.

22. An expression vector comprising a nucleotide sequence encoding an antigen, wherein the expression vector is configured to express the antigen in a modified bacterium or derivative thereof having a reduced number of expressed genes, optionally on the surface of the modified bacterium or derivative thereof.

23. The expression vector of claim 22, comprising an autotransporter (AT) expression vector.

24. The expression vector of claim 22 or claim 23, wherein the vector comprises a codon optimized sequence encoding the antigen.

25. The expression vector of claim 22 or claim 23, wherein the AT expression vector comprises a monomeric vector or a trimeric vector.

26. The expression vector of any one of claims 22-25, wherein the nucleotide sequence encoding the antigen is positioned under control of an inducible promoter or a constitutive promoter.

27. The expression vector of any one of claims 22-26, wherein the antigen is expressed as a monomer or as a trimer.

28. The expression vector of any one of claims 22-27, provided in a pharmaceutically acceptable carrier.
